(19) United States
(12) Patent Application Publication
(10) Pub. No.: US 2018/0306800 A1
(43) Pub. Date: Oct. 25, 2018

(54) METHODS FOR PREDICTING CANCER PATIENT'S CLINICAL RESPONSE TO ANTI-CANCER COMPOUNDS

(71) Applicant: The Regents of the University of Colorado, a body corporate, Denver, CO (US)

(72) Inventors: Xuedong Liu, Niwot, CO (US); Gail Eckhardt, Boulder, CO (US); Gan Zhang, Niwot, CO (US); Conggang Zhang, Boulder, CO (US); Zeyu Liu, Boulder, CO (US); Eric Bunker, Boulder, CO (US)

(21) Appl. No.: 15/959,158

(22) Filed: Apr. 20, 2018

Related U.S. Application Data
(60) Provisional application No. 62/487,677, filed on Apr. 20, 2017.

(51) Int. Cl. G01N 33/574; A61P 35/00; A61K 31/44; A61K 31/495; A61K 31/704; A61K 39/395; A61K 38/15; A61K 31/277; C12Q 1/6886

(52) U.S. Cl. CPC G01N 33/57492; A61P 35/00; A61K 31/44; A61K 31/495; A61K 31/704; C12Q 2600/158; A61K 38/15; A61K 31/277; C12Q 1/6886; C12Q 2600/106; A61K 39/39558

(57) ABSTRACT

Methods and materials involved in assessing samples (e.g., cancer cells) for the status of PINK1-Parkin pathway biomarkers, as well as materials and methods for identifying cancer patients likely to respond to a particular cancer treatment regimen.

### CROSS-REFERENCE TO RELATED APPLICATIONS

This application claims priority under 35 U.S.C. § 119(e) to U.S. Provisional Patent Application Ser. No. 62/487,677, filed Apr. 20, 2017, the entire disclosure of which is incorporated herein by reference.

### STATEMENT OF FEDERALLY SPONSORED RESEARCH

This invention was made with government support under grant number GM113141 awarded by the National Institutes of Health. The government has certain rights in the invention.

### TECHNICAL FIELD

This document relates to methods and materials involved in assessing samples (e.g., cancer cells) for the presence or loss or levels of a biomarker. For example, this document provides methods and materials for determining whether or not a cell (e.g., a cancer cell) contains a level of certain biomarkers. This document also provides materials and methods for identifying cancer patients likely to respond to a particular cancer treatment regimen.

### BACKGROUND OF INVENTION

Cancer is a serious public health problem, with 562,340 people in the United States of America dying of cancer in 2009 alone. American Cancer Society, Cancer Facts & Figures 2009 (available at American Cancer Society website). One of the primary challenges in cancer treatment is discovering relevant, clinically useful characteristics of a patient's own cancer and then, based on these characteristics, administering a treatment plan best suited to the patient's cancer.

Sorafenib (NEXAVAR™) is a drug approved as first-line treatment for advanced hepatocellular carcinoma (HCC), advanced renal cell carcinoma while regorafenib (STIVARGA™), a derivative of sorafenib, was approved as a new treatment option in late-stage metastatic colorectal cancer (CRC). Despite its success entering the market, the overall outcomes for both drugs are still far from satisfactory. As single agent, regorafenib only increases the median overall survival of late-stage metastatic colorectal cancer by about 6 weeks. Clinical trial data for regorafenib suggests that different subgroups of patients may exhibit different responses to treatment (Grothey et al, Lancet 2013, 381(9863):303-12). The variable responses seen with sorafenib and regorafenib have been attributed to genetic heterogeneity of tumors. Valinomycin is a dodecadepsipeptide antibiotic obtained from Streptomyces tsusimaensis and Streptomyces fulvissimus, which acts as a potassium ionophore known to collapse the potassium gradient across the mitochondrial inner membrane and depolarize mitochondria. Carbonyl cyanide m-chlorophenyl hydrazone (CCCP) is a mitochondrial uncoupler protonophore.

Currently there are no credible predictive biomarkers for sensitivity or resistance to sorafenib, regorafenib, valinomycin and/or CCCP for any tumor types. Thus, while strides have been made in the field of personalized medicine, there is still a significant need for better molecular diagnostic tools to characterize patients' cancers.

### SUMMARY

Parkin was initially identified as a gene implicated in autosomal recessive juvenile Parkinsonism. Mutations in the Parkin gene and are known to be associated with early-onset familial autosomal recessive Parkinson's disease (PD). Parkin, [“Parkinson protein 2”; also referred to as PARK2, AR-JP, LPRS2, PDJ, PRKN; Entrez 5071 (Human) 50873 (Mouse); mRNA NM_004562 (Human) NM_016694 (Mouse); protein NP_004553 (Human) NP_057903 (Mouse)] a member of the RING-IBR-RING (RBR) family of ubiquitin E3 ligases, works in tandem with PINK1 [PTEN-induced putative kinase 1; also referred to as Park6 and BRPK; Entrez 65081 (Human) 68943 (Mouse); mRNA NM_032409 (Human) NM_026880 (Mouse); protein NP_115785 (Human) NP_081156 (Mouse)], a mitochondrial Ser/Thr protein kinase, to control mitochondrial homeostasis in response to cellular stress signaling. Besides the well documented association of PINK1 and Parkin in neurodegenerative diseases, this pathway has also been linked to pathogenesis of other human diseases. In particular, Parkin has been implicated as a tumor suppressor protein. Parkin is located on the long arm of chromosome 6, a segment which has long been known to be altered or deleted in a wide variety of human cancers. Loss of the Parkin gene has been reported in a subset of human CRC, HCC, and glioblastoma samples. Parkin knockout mice had enhanced hepatocyte proliferation and developed macroscopic hepatic tumors with the characteristics of hepatocellular carcinoma and resistance to apoptosis induced by cisplatin, doxorubicin and etoposide. These studies demonstrated that Parkin is a tumor suppressor gene and loss of Parkin may be associated with acquired chemoresistance in tumor cells.

The inventors have discovered that sorafenib specifically activates a cellular signaling pathway that renders tumor cells apoptotic. The key target that controls the sorafenib response is Parkin, a ubiquitin E3 ligase that is mutated in familial Parkinson's disease. Recent studies demonstrate that Parkin is frequently deleted or mutated in sporadic colorectal cancer. The inventors showed that cell lines that are deficient for the Parkin pathway are more resistant to cell death induced by sorafenib. Further, Parkin is required by sorafenib to suppress expression of Bcl-2 and Mcl1, both of which are key anti-apoptotic proteins. Based on these mechanistic insights, this disclosure provides rational drug combinations that improve sorafenib apoptotic cell death responses. Thus, in one aspect, this disclosure provides methods of using the presence or activity of the Parkin pathway as a biomarker for identifying the population of cancer patients likely to gain clinical benefit from sorafenib and/or regorafenib.

One aspect of this disclosure provides methods related to predicting a cancer patient's response to a cancer treatment regimen comprising an anti-cancer agent, using the expression levels of the functional biomarkers identified by the inventors as correlated with a cancer patient's likelihood of response (or lack of response) to the cancer treatment regimen. These expression levels may be assessed at the genetic levels (mRNA, including increased transcription, gene duplication/amplification, polysomy, prolonged mRNA transcript half-life, mutations that lead to gain of function, loss of function, or increased or decreased transcription, etc.) and at the protein levels (protein content, protein activity, gain of function or loss of function mutations, prolonged protein half-life, post translational modifications, etc.).

One embodiment of this aspect of the disclosure is a method that includes determining the expression level of a biomarker in a cancer cell from a cancer patient. The biomarker is selected from: high expression levels of PINK1, high expression levels of Parkin, low expression levels of Mcl-1, low expression levels of Bcl-2, and observed mitochondrial localization of wild type Parkin following treatment with sorafenib or regorafenib, in combination with low Mcl-1 or Bcl-2 expression. The biomarker is then correlated with an increased likelihood that the cancer patient will respond to the cancer treatment regimen comprising the anti-cancer agent.

Another embodiment of this aspect of the disclosure is a method that includes determining, in a cancer cell from the cancer patient, the expression level of a biomarker selected from PINK1, Parkin, Mcl-1, and Bcl-2. The level of at least one of PINK1 and Parkin that is greater than a reference number may then be correlated with an increased likelihood that the cancer patient will respond to a treatment regimen including an anti-cancer agent. Similarly, the level of at least one of Mcl-1 and Bcl-2 that is lower than a reference number may then be correlated with an increased likelihood that the cancer patient will respond to a treatment regimen including an anti-cancer agent.

Another embodiment of this aspect of the disclosure is a method that includes determining, in a cancer cell from the cancer patient, the expression level of a biomarker selected from PINK1, Parkin, Mcl-1, Bcl-2, and, correlating the level of at least one of PINK1 and Parkin that is lower than a reference number or correlating the level of at least one of Mcl-1 and Bcl-2 that is greater than a reference number with an increased likelihood that the cancer patient will not respond to a treatment regimen including an anti-cancer agent.

Another embodiment of this aspect of the disclosure is a method that includes determining, in a cancer cell from a cancer patient or genomic DNA obtained therefrom, the level of a biomarker selected from PINK1, Parkin, Mcl-1, Bcl-2, and, administering to the cancer patient a cancer treatment regimen comprising an anti-cancer agent, if the biomarker expression level is similar to a reference number associated with response to the biomarker expression level.

Another embodiment of this aspect of the disclosure is a method that includes selecting a patient for treatment with an anti-cancer agent on the basis of the patient having, in a cancer cell, the level of a biomarker selected from PINK1, Parkin, Mcl-1, Bcl-2, and, selectively administering the anti-cancer agent to the patient.

Another embodiment of this aspect of the disclosure is a method that includes assaying a cancer cell from a subject for the level of a biomarker selected from PINK1, Parkin, Mcl-1, Bcl-2, and, administering an anti-cancer agent to the subject if the cancer cell has a level of at least one of these biomarkers indicative of a cancer that will respond to the anti-cancer agent or administering a composition comprising PINK1, Parkin and Mcl-1 proteins to the subject if the cancer cell does not have a level of at least one of these biomarkers indicative of a cancer that will respond to the anti-cancer agent.

Another embodiment of this aspect of the disclosure is a method that includes obtaining a cancer cell from a patient diagnosed with cancer, the cancer cell comprising nucleic acids from the patient and detecting in the nucleic acids the level of a biomarker selected from PINK1, Parkin, Mcl-1, Bcl-2, and, correlating the level of the biomarker with an increased likelihood for the patient to have a beneficial clinical response to an anti-cancer medication.

In these embodiments, the anti-cancer medication is one or more medications selected from sorafenib, regorafenib, ABT-737, doxorubicin, cetuximab, valinomycin, and CCCP. In certain embodiments, the anti-cancer medication is at least one of sorafenib, and regorafenib. In other embodiments, the anti-cancer medication is at least one of sorafenib and regorafenib combined with at least one of ABT-737, doxorubicin, and cetuximab. In other embodiments, the anti-cancer medication is at least one of valinomycin, and CCCP. In specific embodiments, the anti-cancer medication is valinomycin. In specific embodiments, the anti-cancer medication is CCCP.

In these embodiments, the benefit received from the administration of the anti-cancer medication may be selected from an anti-cancer response to the medication, better disease control rate, longer time to progression, and increased survival following treatment with the anticancer compound.

In these embodiments the cancer cell can be one of a hepatocellular carcinoma cell, a renal cell cancinoma, a pancreatic carcinoma cell, a glioblastoma cell, a colorectal cancer (CRC) cell, a chemotherapy-refractory metastatic CRC cell, and a late-stage metastatic CRC cell.

In these embodiments, the biomarker is detected by a method selected from Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry. In some embodiments, the biomarker is detected by immunohistochemical (IHC) analysis.

Another aspect of this disclosure provides assay kits for selecting a cancer patient who is predicted to benefit or not to benefit from therapeutic administration of an anticancer compound. These assay kits include a means for detecting, in a sample of cancer cells, a level of a biomarker or a combination of biomarkers selected from high expression levels of PINK1, high expression levels of Parkin, low expression levels of Mcl-1, low expression levels of Bcl-2, and observed mitochondrial localization of wild type Parkin following treatment with sorafenib or regorafenib, in combination with low Mcl-1 or Bcl-2 expression. These assay kits also include a means for detecting, in the sample of cancer cells, a control selected from a control sample for detecting sensitivity to the anticancer compound, a control sample for detecting resistance to the anticancer compound. These assay kits also include information containing a predetermined control level of the biomarker that has been correlated with sensitivity to the anticancer compound, and information containing a predetermined control level of the biomarker that has been correlated with resistance to the anticancer compound.

These assay kits may also include at least one means for detecting at least one mutation in the PINK1 or Parkin genes.

In these assay kits, the means for detecting the biomarkers may include a nucleotide probe that hybridizes to a portion of the biomarker. Alternatively or additionally, in these assay kits, the means for detecting the biomarkers may include a detectable label. In certain embodiments, in these assay kits the means for detecting is immobilized on a substrate.

This Summary of the Invention is neither intended nor should it be construed as being representative of the full extent and scope of the present invention. Moreover, references made herein to “the present invention,” or aspects thereof, should be understood to mean certain embodiments of the present invention and should not necessarily be construed as limiting all embodiments to a particular description. The present invention is set forth in various levels of detail in the Summary of the Invention as well as in the attached drawings and the Description of Embodiments and no limitation as to the scope of the present invention is intended by either the inclusion or non-inclusion of elements, components, etc. in this Summary of the Invention. Additional aspects of the present invention will become more readily apparent from the Description of Embodiments, particularly when taken together with the drawings.

### BRIEF DESCRIPTION OF DRAWINGS

FIGS. 1A-1C show that sorafenib induces Parkin mitochondrial relocalization in HeLa cells. (FIGS. 1A-1C) HeLa cells expressing RFP-Smac-MTS and Venus-Parkin were treated with 10 μM CCCP (FIG. 1A) or 20 μM sorafenib (FIG. 1B) for 90 min and 160 min respectively. Both CCCP and sorafenib trigger Parkin mitochondrial recruitment. Scale bar, 10 μm. (FIG. 1C) Quantification of (FIG. 1A) and (FIG. 1B) by Pearson co-localization algorithm using MATLAB.

FIGS. 2A-2H show that sorafenib induces Parkin mitochondrial relocalization and activation in a PINK1dependent manner. FIG. 2A shows PINK1 knockout (PINK1 −/−) MEF cells stably expressing human Venus-Parkin alone or human VenusParkin plus hPINK1 were treated with sorafenib (40 μM). Mitochondrial localization of Venus-Parkin is observed in the PINK1 positive MEF but not PINK1 null cell line. Scale bar, 10 μm. FIG. 2B shows Parkin activation by sorafenib treatment in MEF cells indicated by immunoblotting analysis. The slow migrating band in the Parkin blot corresponds to the S65 phosphorylated and mono-ubiqutinylated Parkin. Ezrin was used as a loading control. FIG. 2C is a time course of Venus-Parkin recruitment to mitochondria upon treatment with sorafenib. At each time, the fraction of cells (%) that have Parkin puncta was quantified and plotted. Error bars, standard deviations. FIG. 2D shows Sorafenib-induced Parkin phosphorylation at Ser65. HeLa cells expression wild type or Parkin S65A mutant were treated with 20 μM sorafenib for indicated time and blotted with the Parkin antibody. GAPDH was used as a loading control. FIG. 2E shows Phosphorylation of Parkin Ser65 is required for sorafenib-induced Parkin activation. HeLa cells expressing RFP-Smac and Venus-Parkin-WT or mTurqoise-ParkinS65A were treated with 20 μM sorafenib for indicated time. Parkin mitochondrial recruitment is perturbed by Ser65 mutation. FIG. 2F shows HeLa cells expressing both Venus-Parkin and mTurqoiseParkinS65A were treated with sorafenib for indicated time and mitochondrial recruitment was measured. Scale bar, 10 μm. FIGS. 2G and 2H show quantitation of Parkin mitochondria recruitment at 2 h following sorafenib treatment. (n=6; *p<0.0001, student's unpaired t test).

FIGS. 3A-3C show that sorafenib stabilizes the endogenous PINK1 and ectopically expressing PINK1-EGFP. FIG. 3A shows HeLa cells treated with 30 μM sorafenib for indicated time were blotted with the PINK1 and GAPDH antibodies. FIG. 3B is a time course of PINK1-EGFP induction in HeLa cells stably expressing PINK1-EGFP upon exposure to 30 μM sorafenib for indicated time. FIG. 3C shows live cell imaging of PINK1-EGFP in HeLa cells treated with 30 μM sorafenib for 3.5 h. The nuclei were identified by Hoechst 33258 staining.

FIGS. 4A-4K show that sorafenib inhibits both complex II/III and complex V and causes rapid depolarization of mitochondria membrane potential. FIG. 4A shows the mitochondrial membrane potential of HeLa cells was measured using a chemical probe, tetramethyl-rhodamine, methyl ester (TMRE). Cells were treated with DMSO, 10 μM CCCP, and 20 μM sorafenib for 40 min. The mitochondrial membrane potential was quantified by measuring the TMRE intensity in over the 40 min time course. Both CCCP and sorafenib treatments cause rapid loss of mitochondrial membrane potential while no significant change was observed for DMSO treatment. FIG. 4B shows that sorafenib inhibits mitochondrial electron transport activity. Cells growing on a glass bottom 96 well plate were scanned for 2 min with 454 nm excitation wavelength and emission between 505 and 550 nm was collected to establish the baseline autofluoresence for normalization. Cells were treated with either DMSO, 20 μM sorafenib, or 10 μM antimycin A and scanned for 2 min prior to the addition of 1 μM of CCCP and scanned for additional 2 min. Relative FAD autofluoresence for each treatment was plotted. Error bars indicate standard deviation. FIGS. 4C-4F show the activities of sorafenib against mitochondrial complex I-V in vitro using the methods described in Experimental Procedures. Negative control (DMSO), positive control and sorafenib were tested for inhibition at indicated doses and relative activity of each measurement was plotted. Error bars indicate standard deviation. A student's unpaired t test was performed to determine the significance of the observed inhibitory activity of the compounds. The p values for each test are as follows: Complex I, DMSO vs Rotenone p=0.005; DMSO vs Sorafenib p=0.1807. Complex DMSO vs antimycin p=0.0091; DMSO vs sorafenib p=0.0065. Complex IV, DMSO vs KCN p=0.017; DMSO vs sorafenib p=0.1375. Complex V, DMSO vs oligomycin p=1.81×10−5; DMSO vs sorafenib p=1.47×10−4. FIGS. 4G and 4H are dose response inhibitory activity of sorafenib towards complex II/III and complex V. IC50 values were calculated in MATLAB by fitting dose-response curves. FIG. 4I shows dual inhibition of complex II/III and complex V is necessary and sufficient to activate Parkin mitochondrial recruitment. HeLa Venus-Parkin/RFP-Smac cells were treated with indicated concentrations of negative control (DMSO), positive control (CCCP, 10 μM), sorafenib (20 μM), antimycin (10 μM), oligomycin (2.5 μM) or antimycin+oligomycin at a dose of 10 μM and 2.5 μM for 2 h and imaged. Hoechst 33258 was used to count cell nuclei. FIG. 4J shows quantification of Parkin mitochondrial recruitment by Pearson co-localization algorithm using MATLAB. Error bars, standard deviations. FIG. 4K is a quantitative measurement of the mitochondrial membrane potential using TMRE in HeLa cells treated with negative control (DMSO), positive control (CCCP, 10 μM), sorafenib (20 μM), antimycin (10 μM), oligomycin (2.5 μM), KCN (10 μM), KCN+oligomycin (10 μM and 2.5 μM) or antimycin+oligomycin (10 μM and 2.5 μM) at a dose of 10 μM for 3 h. Images were taken every five minutes. Error bars, standard deviations.

FIGS. 5A-5H show CCCP- and sorafenib-induced different cell fate outcomes in a Parkin-dependent manner. FIGS. 5A and 5B show that CCCP induces Parkin dependent mitophagy but sorafenib does not. HeLa cells expressing VenusParkin-WT, RFP-Smac, and CFP-LC3 were treated with 10 μM CCCP and 20 μM sorafenib for indicated time. Mitophagy response was visualized with fluorescent microscopy exhibiting translocation of Parkin and LC3 to the mitochondria and quantified by collecting image pairs and analyzed via MATLAB to detect colocalization (FIG. 5C). CCCP induced robust LC3 and mitochondria colocalization but not sorafenib. FIG. 5D shows the apoptosis response was visualized with fluorescent microscopy exhibiting RFP-Smac release from mitochondria and quantified. Sorafenib induced strong apoptosis but not CCCP. FIGS. 5E-5H show sorafenib-induced apoptosis was dependent on Parkin. FIGS. 5E and 5F show HeLa cells expressing RFP-Smac and Venus-Parkin-WT or Venus-Parkin-T240R were incubated with 30 μM sorafenib in a 7.5-hour time manner. Apoptosis was quantified by RFP-Smac release. Scale bar, 10 μm. FIG. 5G shows the apoptotic cell number of HeLa cells, HeLa cells expressing Venus-Parkin-WT, and HeLa cells expressing Venus-Parkin-T240R was quantified by RFP-Smac releasing from mitochondria. 100 cells were quantified for each cell line for indicated time points. FIG. 5H shows the quantification of apoptotic percentages of HeLa cells expressing wild-type or T240R mutant Parkin.

FIGS. 6A-6J show that sorafenib, but not CCCP, induces Parkin-dependent suppression of Mcl-1 and activates apoptotic initiator caspases (FIGS. 6A and 6B) HeLa Parkin-WT or Parkin-T240R cells were exposed to 20 μM sorafenib (FIG. 6A) or 10 μM CCCP in a 6-hour time course. Sorafenib induces robust PARP cleavage and activation of initiator caspase-3 characterized by reduction of pro-caspase signal. The effect of CCCP on PARP or pro-caspase-3 cleavage is modest. The effect of sorafenib was not observed in the mutant Parkin cell line. FIGS. 6C and D show the effect of sorafenib on anti-apoptotic Bcl-2 family of proteins were monitored by immunoblotting. Sorafenib causes a depletion of Mcl-1 but not Bcl-xL or Bcl-2 in a Parkin dependent manner. FIG. 6E shows that sorafenib and CCCP have similar effects on PINK1 stabilization and Parkin activation but differ in Mcl-1 suppression. FIG. 6F shows suppression of Mcl-1 by sorafenib can be reversed by proteasome inhibitor MG132. FIG. 6G is immunoblotting showing the suppression of endogenous Mcl-1 expression by Mcl-1 shRNA. HeLa cells expressing VenusParkin and RFP-Smac were infected with shRNA for Mcl-1. FIG. 6H shows that CCCP induces apoptosis in HeLa cells with reduced Mcl-1 expression. Sorafenib induced stronger apoptosis in Mcl-1 knockdown cells. Apoptotic cell death was quantified by counting cells positive for RFP-Smac release. FIGS. 6I and 6J show overexpression of CFP tagged mouse Bcl-2 in HeLa Parkin cells suppresses sorafenib induced apoptosis. Immunblotting confirms the ectopic expression of CFP-mBcl-2. Quantification of apoptotic percentages of HeLa Parkin cells and HeLa Parkin mBcl-2 cells upon incubation with 20 μM sorafenib.

FIGS. 7A-7C shows that sorafenib and Bcl-2 inhibitor act in combination to induce switch of autophagy to apoptosis. FIG. 7A shows sorafenib-induced Parkin-dependent cell death can be affected by Bcl2 protein expression. Combination of ABT-737 with sorafenib synergistically triggers robust apoptosis. Cells were treated with 30 μM sorafenib or 10 μM ABT-737 or both. FIG. 7B shows CCCP can induce cellular apoptosis in combination with ABT-737. ABT 737, a selective inhibitor of Bcl-2, could not trigger cell death by itself. However, combination of 10 μM CCCP and ABT 737 switch autophagy to apoptosis. FIG. 7C is a proposed model for sorafenib induced cellular response via kinase dependent or mitochondrial dependent pathways.

FIGS. 8A-8C show high content screening (HCS) of the FDA approved oncology drug library for compounds that can regulate Parkin mitochondrial recruitment. FIGS. 8A and 8B show HeLa cells expressing RFP-Smac MTS and Venus-Parkin-WT were treated with 20 μM sorafenib. The Parkin puncta (light) and mitochondrial puncta (dark) were imaged. The colocalization of Parkin with mitochondria was quantified using a MetaXpress Transfluor-Colocalization Application Module (Molecular Devices). FIG. 8A shows the Parkin translocation to Mitochondria is significant at 1.5 h treated with 20 μM sorafenib. FIG. 8B shows colocalization of Parkin and mitochondria is strong when cells were treated with 20 μM sorafenib at 3 h. In contrast, cells that were treated with 20 μM Raloxifene HCl showed almost none Parkin mitochondrial translocation. Scale bar, 10 μm. FIG. 8C is a heat map of the colocalization quantification of HeLa cells treated with 20 μM drugs of the FDA proved drug library (101 compounds, seen in supplementary drug table). Two cell lines were used for the quantification. One is HeLa cell line expressing Venus-Parkin-WT and RFP-Smac, and the other is HeLa cell line expressing Venus-ParkinT240R and RFP-Smac. Each drug (20 μM) was incubated with these two HeLa cell lines separately for 1.5 h, and Parkin and mitochondria colocalization was quantified automatically. Sorafenib is the only hit that triggers Parkin mitochondrial localization.

FIGS. 9A-9E show that sorafenib induces apoptosis in a PINK1-dependent manner FIG. 9A shows that sorafenib induces caspase-dependent apoptotsis in HeLa Venus-Parkin cells. HeLa cells expressing Venus-Parkin-WT and stained with Hoechst 33258 were treated with 25 μM sorafenib in the presence or absence of 20 μM Z-VAD in a 12-hour time course. Cells were incubated with sorafenib for 1.5 h prior to image capture. Scale bar, 10 μm. FIG. 9B shows quantification of cell death at 10 h. (n=3; *p<0.0001, student's unpaired t test). FIG. 9C shows PINK1 knockdown decreased sorafenib induced apoptosis in HeLa cells expressing Venus-Parkin. FIG. 9D shows HeLa cells expressing Venus-Parkin-WT were transfected with Control or PINK1 siRNA and treated with 25 μM sorafenib in a 14-hour time course. Cells were incubated with sorafenib for 1.5 h prior to image capture. Apoptotic cell death was visualized by staining with Hoechst 33258. Quantitation of cell death was performed using an automated cell heath application module in MetaXpress software. Representative images are shown. Scale bar, 10 μm. Immunoblotting showed PINK1 knockdown effect. FIG. 9E is quantification of cell death at 14 h. (n=3; *p<0.0001, student's unpaired t test).

FIGS. 10A and 10B show the restoration of PINK1 and Parkin in MEF null cells increase apoptosis response to sorafenib. FIGS. 10A and 10B show PINK1 null MEFS, Parkin null MEFs, and PINK1 null MEF cells expressing human PINK1 (hPINK1) and human Parkin (hParkin) were treated with 25 μM sorafenib for 10 h. Apoptosis was monitored by NucView caspase-3 sensor staining. MEFs with human PINK1/Parkin induced much stronger apoptosis. Scale bar, 10 μm.

### DETAILED DESCRIPTION

This document provides methods and materials involved in assessing samples (e.g., cancer cells) for the status of biomarkers indicative of cancer patient response to anti-cancer therapeutic regimens.

In general, an analysis of the PINK1-Parkin signaling pathway components can reveal whether a cancer cell is likely sensitive or resistant to treatment with certain anti-cancer compounds. Thus, the present invention is generally related to the identification of cancer patients that are predicted to benefit from the therapeutic administration of an anti-cancer compound. The present invention is also generally related to methods to identify treatments that can improve the responsiveness of sorafenib/regorafenib-resistant cancer cells to the treatment, and to the development of adjuvant treatments that enhance the sorafenib response.

Accordingly, one embodiment of the present invention relates to a method and corresponding assay kit for use to select a cancer patient who is predicted to benefit from therapeutic administration of an anticancer compound. The method generally includes detecting in a sample of cancer cells from a patient the biomarkers related to the PINK1-Parkin pathway that have been discovered by the inventors to be invaluable in the detection of anticancer-sensitive or resistant cells, thus predicting the patients' clinical benefit to treatment using these anticancer compounds. Based on the inventors' discovery, a variety of tests and combinations of biomarker detection strategies are proposed, and will be discussed in detail below. Initially, however, the present invention includes the use of the following strategies for detection of biomarkers, alone or in various combinations: (1) detection of the expression level of PINK1; (2) detection of the expression level of Parkin; (3) detection of the expression level of Mcl-1; (3) detection of the expression level of Bcl-2; and 4) the observed mitochondrial localization of wild type Parkin following treatment with sorafenib or regorafenib, in combination with low Mcl-1 or Bcl-2 expression. The invention includes the use of these detection protocols individually or in various combinations, and the invention further includes the use of various combinations of one or more biomarker detection techniques to further enhance the ability of the present method to identify sensitive and resistant cancer cells, as well as to predict patients' clinical benefit (e.g., response and outcome) to certain anticancer compounds. The inventors have also discovered that combinations of the tests described herein can be used to select patients with cancer, who will not have clinical benefit from certain anticancer compounds.

The anticancer compounds useful in the methods of the invention, herein referred to as “anticancer compounds of the invention” include one or more of sorafenib, regorafenib, ABT-737, doxorubicin, cetuximab, valinomycin, and carbonyl cyanide m-chlorophenyl hydrazone (CCCP).

The present inventors have discovered that patients with cancer cells displaying high levels of PINK1 and/or Parkin proteins with respect to normal or absent expression levels are predicted to be responsive to treatment with of at least one of anticancer compounds of the invention. Similarly, patients with cancer cells displaying low levels of Bcl2 and/or Mcl-1 proteins with respect to normal or high expression levels are predicted to be responsive to treatment with of at least one anticancer compounds of the invention.

Levels of protein expression of PINK1 and/or Parkin may be assessed by immunohistochemistry and high levels are statistically significantly associated with better response, disease control rate, time to progression and survival following treatment with these anticancer compounds.

The methods and test kits provided by the present invention are extremely useful for patients with any cancer that can be treated with at least one anticancer compounds of the invention, such as, hepatocellular carcinoma, renal cell carcinoma, pancreatic carcinoma, glioblastoma, colorectal cancer (CRC), chemotherapy-refractory metastatic CRC, and late-stage metastatic CRC. Such patients might, as a result of the methods provided herein, be spared from side effects and financial costs of an ineffective therapy in the event that they do not cancer with elevated PINK1 or Parkin or low levels of Mcl-1 or Bcl-2. Second, it is useful for physicians, who can recommend, or not, specific treatments using these anticancer compounds to particular patients based on information on the characteristics of their cancer cells.

Thus, in one or more embodiments, the methods of the invention include the detection in a sample of cancer cells from a patient a level of PINK1 or Parkin or Mcl-1 or Bcl-2 protein expression (e.g., by using immunohistochemical techniques). Patients with cancer cells displaying higher levels of PINK1 or Parkin protein are predicted to be responsive to treatment with an anticancer compound of the invention, and are therefore the best candidates for the use of this line of therapy. Patients having cancer cells with high PINK1 and/or Parkin protein expression in combination with low Mcl-1 and/or Bcl-2 expression, are predicted to be responsive to treatment with a cancer compound of the invention. Patients having cancer cells in which treatment with at least one of sorafenib and regorafenib mitochondrial localization of wild type Parkin in combination with low Mcl-1 or Bcl-2 expression are predicted to be responsive to treatment with a cancer compound of the invention.

In one or more embodiments of the invention, the methods include the detection of PINK1 or Parkin or Mcl-1 or Bcl-2 proteins using immunohistochemistry (IHC) techniques.

It will be apparent to those of skill in the art from this disclosure that a variety of combinations of the above-described biomarkers and detection protocols can enhance or improve the ability to identify patients that are predicted to be responsive to therapy with an anticancer compound of the invention (and patients that are predicted to be poor responders). Therefore, any combination of the use of the biomarkers, detection protocols and detection techniques is encompassed by the invention. Moreover, the invention is not limited to the detection techniques described herein (e.g., IHC), since other techniques may be used to achieve the same result.

The methods of the present invention include detecting in a sample of cancer cells from a patient to be tested, any one or any combination of 2, 3, 4, 5, 6 or all 7 of the following biomarkers and types of detection of such biomarkers: (1) PINK1 protein expression; (2) Parkin protein expression; (3) Mcl-1 protein expression; (4) Bcl-2 protein expression; and/or (5) mitochondrial localization of wild type Parkin in combination with low Mcl-1 or Bcl-2 expression.

Suitable methods of obtaining a cell sample from a patient are known to a person of skill in the art. A patient sample can include any bodily fluid or tissue from a patient that may contain cancer cells or proteins of cancer cells. More specifically, according to the present invention, the term “test sample” or “patient sample” can be used generally to refer to a sample of any type which contains cells or products that have been secreted from cells to be evaluated by the present method, including but not limited to, a sample of isolated cells, a tissue sample and/or a bodily fluid sample. Most typically in the present invention, the sample is a tissue sample. According to the present invention, a sample of cancer cells is a specimen of cells, typically in suspension or separated from connective tissue which may have connected the cells within a tissue in vivo, which have been collected from an organ, tissue or fluid by any suitable method which results in the collection of a suitable number of cells for evaluation by the method of the present invention. The cells in the cell sample are not necessarily of the same type, although purification methods can be used to enrich for the type of cells that are preferably evaluated. Cells can be obtained, for example, by scraping of a tissue, processing of a tissue sample to release individual cells, or isolation from a bodily fluid.

A tissue sample, although similar to a sample of isolated cells, is defined herein as a section of an organ or tissue of the body which typically includes several cell types and/or cytoskeletal structure which holds the cells together. One of skill in the art will appreciate that the term “tissue sample” may be used, in some instances, interchangeably with a “cell sample”, although it is preferably used to designate a more complex structure than a cell sample. A tissue sample can be obtained by a biopsy, for example, including by cutting, slicing, or a punch.

A bodily fluid sample, like the tissue sample, contains the cells to be evaluated, and is a fluid obtained by any method suitable for the particular bodily fluid to be sampled. Bodily fluids suitable for sampling include, but are not limited to, blood, mucous, seminal fluid, saliva, sputum, bronchial lavage, breast milk, bile and urine.

In general, the sample type (i.e., cell, tissue or bodily fluid) is selected based on the accessibility and structure of the organ or tissue to be evaluated for tumor cell growth and/or on what type of cancer is to be evaluated. For example, if the organ/tissue to be evaluated is the colon, the sample can be a sample of epithelial cells from a biopsy (i.e., a cell sample) or a colon tissue sample from a biopsy (a tissue sample). The present invention is particularly useful for evaluating patients with lung cancer and particularly, colorectal carcinoma, and in this case, a typical sample is a section of a colon tumor from the patient.

The protein expression in cancer cells according to the invention can be measured, for example in immunohistochemistry assays, in tumor cell nuclei, cytoplasm and/or membranes. Immunohistochemistry, as well as other detection methods, can be performed in primary tumors, metastatic tumors, locally recurring tumors, sputum, bronchial lavage, ascites, spinal fluid, or other tumor settings. The markers can be measured in tumor specimens that are fresh, frozen, fixed or otherwise preserved.

Once a sample is obtained from the patient, the sample is evaluated for detection of one or more of any of the biomarkers described herein. In some embodiments of the present invention, a tissue, a cell or a portion thereof (e.g., a section of tissue, a component of a cell such as nucleic acids, etc.) is contacted with one or more nucleic acids. Such protocols are used to detect gene expression, gene amplification, and/or gene polysomy, for example. Such methods can include cell-based assays or non-cell-based assays. The tissue or cell expressing a target gene is typically contacted with a detection agent (e.g., a probe, primer, or other detectable marker), by any suitable method, such as by mixing, hybridizing, or combining in a manner that allows detection of the target gene by a suitable technique.

The patient sample is prepared by any suitable method for the detection technique utilized. In one embodiment, the patient sample can be used fresh, frozen, fixed or otherwise preserved. For example, the patient tumor cells can be prepared by immobilizing patient tissue in paraffin. The immobilized tissue can be sectioned and then contacted with a probe for detection of hybridization of the probe to a target gene.

In a preferred embodiment, detection of a nucleic acid according to the present invention is accomplished using hybridization assays. Nucleic acid hybridization simply involves contacting a probe (e.g., an oligonucleotide or larger polynucleotide) and target nucleic acid under conditions where the probe and its complementary target can form stable hybrid duplexes through complementary base pairing. As used herein, hybridization conditions refer to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al. is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284. Nucleic acids that do not form hybrid duplexes are washed away from the hybridized nucleic acids and the hybridized nucleic acids can then be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches.

High stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). One of skill in the art can use the formulae in Meinkoth, supra, to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6×SSC (0.9 M Na+) at a temperature of between about 20° C. and about 35° C., more preferably, between about 28° C. and about 40° C., and even more preferably, between about 35° C. and about 45° C. In particular embodiments, stringent hybridization conditions for DNA:RNA hybrids include hybridization at an ionic strength of 6×SSC (0.9 M Na+) at a temperature of between about 30° C. and about 45° C., more preferably, between about 38° C. and about 50° C., and even more preferably, between about 45° C. and about 55° C. These values are based on calculations of a melting temperature for molecules larger than about 100 nucleotides, 0% formamide and a G+C content of about 40%. Alternatively, Tm can be calculated empirically as set forth in Sambrook et al., supra, pages 9.31 to 9.62.

The hybridized nucleic acids are detected by detecting one or more labels attached to the sample nucleic acids. The labels may be incorporated by any of a number of means well known to those of skill in the art. Detectable labels suitable for use in the present invention include any composition detectable by spectroscopic, photochemical, biochemical, immunochemical, electrical, optical or chemical means. Useful labels in the present invention include fluorescent dyes (e.g., fluorescein, Texas red, rhodamine, Alexa fluors, Spectrum dyes, and the like), quantum dots, radiolabels (e.g., 3H, 125I, 35S, 14C, or 32P), and colorimetric labels. Means of detecting such labels are well known to those of skill in the art. Thus, for example, radiolabels may be detected using photographic film or scintillation counters, fluorescent markers may be detected using a photodetector to detect emitted light and fluorescence microscopes. Colorimetric labels are detected by simply visualizing the colored label.

In accordance with the present invention, an isolated polynucleotide, or an isolated nucleic acid molecule, is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the nucleic acid molecule has been purified, but indicates that the molecule does not include an entire genome or an entire chromosome in which the nucleic acid molecule is found in nature. Polynucleotides such as those used in a method of the present invention to detect genes (e.g., by hybridization to a gene) are typically a portion of the target gene that is suitable for use as a hybridization probe or PCR primer for the identification of a full-length gene (or portion thereof) in a given sample (e.g., a cell sample). An isolated nucleic acid molecule can include a gene or a portion of a gene (e.g., the regulatory region or promoter). An isolated nucleic acid molecule that includes a gene is not a fragment of a chromosome that includes such gene, but rather includes the coding region and regulatory regions associated with the gene, but no additional genes naturally found on the same chromosome. An isolated nucleic acid molecule can also include a specified nucleic acid sequence flanked by (i.e., at the 5′ and/or the 3′ end of the sequence) additional nucleic acids that do not normally flank the specified nucleic acid sequence in nature (i.e., heterologous sequences). Isolated nucleic acid molecule can include DNA, RNA (e.g., mRNA), or derivatives of either DNA or RNA (e.g., cDNA). Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein. Preferably, an isolated nucleic acid molecule of the present invention is produced using recombinant DNA technology (e.g., polymerase chain reaction (PCR) amplification, cloning) or chemical synthesis.

According to the present invention, a probe (oligonucleotide probe) is a nucleic acid molecule which typically ranges in size from about 50-100 nucleotides to several hundred nucleotides to several thousand nucleotides in length. Therefore, a probe can be any suitable length for use in an assay described herein, including any length in the range of 50 to several thousand nucleotides, in whole number increments. Such a molecule is typically used to identify a target nucleic acid sequence in a sample by hybridizing to such target nucleic acid sequence under stringent hybridization conditions. Hybridization conditions have been described in detail above.

PCR primers are also nucleic acid sequences, although PCR primers are typically oligonucleotides of fairly short length (e.g., 8-30 nucleotides) that are used in polymerase chain reactions. PCR primers and hybridization probes can readily be developed and produced by those of skill in the art, using sequence information from the target sequence. (See, for example, Sambrook et al., supra).

In the method of the invention, the level of protein expression in the cancer cell sample is compared to a control level of that protein expression selected from: (i) a control level that has been correlated with sensitivity to an anticancer compound of the invention; and (ii) a control level that has been correlated with resistance to an anticancer compound of the invention. A patient is selected as being predicted to benefit from therapeutic administration of the anticancer compound of the invention, an agonist thereof, or a drug having substantially similar biological activity, if the level of protein expression in the patient's cancer cells is statistically similar to the control level of protein expression that has been correlated with sensitivity to the anticancer compound, or if the level of PINK1 or Parkin protein expression in the patient's cancer cells is statistically greater than the level of that protein expression that has been correlated with resistance to the anticancer compound. A patient is predicted to not benefit from therapeutic administration of an anticancer compound of the invention, an agonist thereof, or a drug having substantially similar biological activity as an anticancer compound of the invention, if the level of PINK1 or Parkin protein expression in the patient's cancer cells is statistically less than the control level of protein expression that has been correlated with sensitivity to the anticancer compound, or if the level of PINK1 or Parkin in the patient's cancer cells is statistically similar to or less than the level of PINK1 or Parkin that has been correlated with resistance to the anticancer compound of the invention.

Alternatively, in other methods of the invention, a patient is selected as being predicted to benefit from therapeutic administration of the anticancer compound of the invention, an agonist thereof, or a drug having substantially similar biological activity, if the level of protein expression in the patient's cancer cells is statistically similar to the control level of protein expression that has been correlated with sensitivity to the anticancer compound, or if the level of Mcl-1 or Bcl-2 protein expression in the patient's cancer cells are statistically lower than the level of that protein expression that has been correlated with resistance to the anticancer compound. A patient is predicted to not benefit from therapeutic administration of an anticancer compound of the invention, an agonist thereof, or a drug having substantially similar biological activity as an anticancer compound of the invention, if the level of Mcl-1 or Bcl-2protein expression in the patient's cancer cells is statistically greater than the control level of protein expression that has been correlated with sensitivity to the anticancer compound, or if the level of Mcl-1 or Bcl-2 in the patient's cancer cells is statistically similar to or greater than the level of Mcl-1 or Bcl-2 that has been correlated with resistance to the anticancer compound of the invention.

According to the present invention, a “control level” is a control level of protein expression, which can include a level that is correlated with sensitivity to the anticancer compounds of the invention or that is correlated with resistance to anticancer compounds of the invention. Therefore, it can be determined, as compared to the control or baseline level of protein expression, whether a patient sample is more likely to be sensitive to or resistant to the anticancer compound therapy (e.g., a good responder or responder (one who will benefit from the therapy), or a poor responder or non-responder (one who will not benefit or will have little benefit from the therapy)).

The method for establishing a control level of expression is selected based on the sample type, the tissue or organ from which the sample is obtained, and the status of the patient to be evaluated. Preferably, the method is the same method that will be used to evaluate the sample in the patient. In a preferred embodiment, the control level is established using the same cell type as the cell to be evaluated. In a preferred embodiment, the control level is established from control samples that are from patients or cell lines known to be resistant or sensitive to an anticancer compound of the invention. In one aspect, the control samples are obtained from a population of matched individuals. According to the present invention, the phrase “matched individuals” refers to a matching of the control individuals on the basis of one or more characteristics which are suitable for the type of cell or tumor growth to be evaluated. For example, control individuals can be matched with the patient to be evaluated on the basis of gender, age, race, or any relevant biological or sociological factor that may affect the baseline of the control individuals and the patient (e.g., preexisting conditions, consumption of particular substances, levels of other biological or physiological factors). To establish a control level, samples from a number of matched individuals are obtained and evaluated in the same manner as for the test samples. The number of matched individuals from whom control samples must be obtained to establish a suitable control level (e.g., a population) can be determined by those of skill in the art, but should be statistically appropriate to establish a suitable baseline for comparison with the patient to be evaluated (i.e., the test patient). The values obtained from the control samples are statistically processed using any suitable method of statistical analysis to establish a suitable baseline level using methods standard in the art for establishing such values.

It will be appreciated by those of skill in the art that a control level need not be established for each assay as the assay is performed but rather, a baseline or control can be established by referring to a form of stored information regarding a previously determined control level for sensitive and resistant patients (responders and non-responders), such as a control level established by any of the above-described methods. Such a form of stored information can include, for example, but is not limited to, a reference chart, listing or electronic file of population or individual data regarding sensitive and resistant tumors/patients, or any other source of data regarding control level that is useful for the patient to be evaluated.

In one embodiment of the present invention, the method includes a step of detecting the expression of a protein. Protein expression can be detected in suitable tissues, such as tumor tissue and cell material obtained by biopsy. For example, the patient tumor biopsy sample, which can be immobilized, can be contacted with an antibody, an antibody fragment, or an aptamer, that selectively binds to the protein to be detected, and determining whether the antibody, fragment thereof or aptamer has bound to the protein. Protein expression can be measured using a variety of methods standard in the art, including, but not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry. In a preferred embodiment, immunohistochemical (IHC) analysis is used to detect protein expression. IHC methods and preferred assessment criteria for detection of protein expression are described in detail, for example, in Hirsch et al., J. Clin. Oncol. 2003, 21:3798-3807,

In one embodiment of the present invention, the method includes an additional step of detection of a mutation in the PINK1 or Parkin genes. In particular, the detection of one or more mutations that reduce or eliminate function of the PINK1 or Parkin proteins. The present invention contemplates the detection of such mutations in the tumor cell samples for use in combination with or as a secondary screening subsequent to the screening for PINK1 or Parkin expression. Detection of one or more mutations in these genes is predictive that a patient is less likely to respond or benefit from administration of an anticancer compound of the invention. Detection of no mutations is predictive that a patient is likely to respond or benefit from administration of an anticancer compound of the invention. Methods for screening for gene mutations are well-known in the art, are described in Lynch et al. and Paez et al., and include, but are not limited to, hybridization, polymerase chain reaction, polyacrylamide gel analysis, chromatography or spectroscopy, and can further include screening for an altered protein product encoded by the gene (e.g., via immunoblot (e.g., Western blot), enzyme-linked immunosorbant assay (ELISA), radioimmunoassay (RIA), immunoprecipitation, immunohistochemistry, immunofluorescence, fluorescence activated cell sorting (FACS) and immunofluorescence microscopy).

The steps of detection of the biomarkers according to the present invention may be combined in many different combinations as described herein, and the steps can be performed in any order, or substantially simultaneously. Statistical analysis to determine differences between controls and patient samples can be performed using any methods known in the art, including, but not limited to, Fisher's exact test of Pearson's chi-square test for qualitative variables, and using Student's t test or analysis of variance for continuous variables. Statistical significance is typically defined as p<0.05.

An agonist, as used herein, is a compound that is characterized by the ability to agonize (e.g., stimulate, induce, increase, enhance, or mimic) the biological activity of a naturally occurring or reference protein or compound. More particularly, an agonist can include, but is not limited to, a compound, protein, peptide, antibody, or nucleic acid that mimics or enhances the activity of the natural or reference compound, and includes any homologue, mimetic, or any suitable product of drug/compound/peptide design or selection which is characterized by its ability to agonize (e.g., stimulate, induce, increase, enhance) the biological activity of a naturally occurring or reference compound. In contrast, an antagonist refers to any compound which inhibits (e.g., antagonizes, reduces, decreases, blocks, reverses, or alters) the effect of a naturally occurring or reference compound as described above. More particularly, an antagonist is capable of acting in a manner relative to the activity of the reference compound, such that the biological activity of the natural or reference compound, is decreased in a manner that is antagonistic (e.g., against, a reversal of, contrary to) to the natural action of the reference compound. Such antagonists can include, but are not limited to, any compound, protein, peptide, or nucleic acid (including ribozymes and antisense) or product of drug/compound/peptide design or selection that provides the antagonistic effect.

Agonists and antagonists that are products of drug design can be produced using various methods known in the art. Various methods of drug design, useful to design mimetics or other compounds useful in the present invention are disclosed in Maulik et al., 1997, Molecular Biotechnology: Therapeutic Applications and Strategies, Wiley-Liss, Inc., which is incorporated herein by reference in its entirety. An agonist or antagonist can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the similar building blocks) or by rational, directed or random drug design. See for example, Maulik et al., supra.

A drug having substantially similar biological activity in the anticancer compound of the invention refers to a drug having substantially any function(s) exhibited or performed by the reference compounds that is ascribed to the reference compounds as measured or observed in vivo (i.e., under physiological conditions) or in vitro (i.e., under laboratory conditions).

Once treated for a particular period of time (e.g., between one to six months), the patient can be assessed to determine whether or not the treatment regimen has an effect. If a beneficial effect is detected, the patient can continue with the same or a similar cancer treatment regimen. If a minimal or no beneficial effect is detected, then adjustments to the cancer treatment regimen can be made. For example, the dose, frequency of administration, or duration of treatment can be increased. In some cases, additional anti-cancer agents can be added to the treatment regimen or a particular anti-cancer agent can be replaced with one or more different anti-cancer agents. The patient being treated can continue to be monitored as appropriate, and changes can be made to the cancer treatment regimen as appropriate.

In some cases, one or more clinicians or medical professionals can obtain a cancer cell sample from a patient and provide that sample to a testing laboratory having the ability to assess the expression levels of cancer cells of the cancer cell sample to provide an indication about the presence or absence of a biomarker level as described herein. In such cases, the one or more clinicians or medical professionals can determine if a patient contains cancer cells having a high or low expression level of the biomarker in the patient by receiving that information about the patient directly or indirectly from the testing laboratory. For example, a testing laboratory, after assessing the expression level of biomarkers from cancer cells as described herein, can provide a clinician or medical professional with, or access to, a written, electronic, or oral report or medical record that provides an indication about the resistance were sensitivity to an anticancer compound of the invention for a particular patient being assessed. Such a written, electronic, or oral report or medical record can allow the one or more clinicians or medical professionals to determine if a particular patient being assessed contains cancer cells sensitive or resistant to an anticancer compound of the invention.

Another embodiment of the invention includes an assay kit for performing any of the methods of the present invention. The assay kit can include any one or more of the following components: (a) a means for detecting in a sample of cancer cells a level of expression of the PINK1 or Parkin gene and/or protein; (b) a means for detecting in a sample of tumor cells a level of expression of the Mcl-1 or Bcl-2 gene and/or protein; (c) a means for detecting in a sample of tumor cells mitochondrial localization of wild type Parkin following treatment with Sorafenib or Regorafenib; (d) a means for detecting in a sample of tumor cells at least one (but can include more than one) mutations in the PINK1 or Parkin gene(s). The assay kit preferably also includes one or more controls. The controls could include: (i) a control sample for detecting sensitivity to the anticancer compounds of the invention being evaluated for use in a patient; (ii) a control sample for detecting resistance to the anticancer compounds of the invention; (iii) information containing a predetermined control level of particular biomarker to be measured with regard to sensitivity or resistance to an anticancer compound of the invention (e.g., a predetermined control level of gene expression and/or protein expression that has been correlated with sensitivity to or resistance to the anticancer compound).

In one embodiment, a means for detecting gene or protein expression can generally be any type of reagent that can be used in a method of the present invention. Such a means for detecting include, but are not limited to: a probe or primer(s) that hybridizes under stringent hybridization conditions to a PINK1 or Parkin gene. Additional reagents useful for performing an assay using such means for detection can also be included, such as reagents for performing in situ hybridization, reagents for detecting fluorescent markers, reagents for performing polymerase chain reaction, etc.

In another embodiment, a means for detecting PINK1, Parkin. Mc1-1 or Bcl-2 protein expression can generally be any type of reagent that can be used in a method of the present invention. Such a means for detection includes, but is not limited to, antibodies and antigen binding fragments thereof, peptides, binding partners, aptamers, enzymes, and small molecules. Additional reagents useful for performing an assay using such means for detection can also be included, such as reagents for performing immunohistochemistry or another binding assay.

The means for detecting of the assay kit of the present invention can be conjugated to a detectable tag or detectable label. Such a tag can be any suitable tag which allows for detection of the reagents used to detect the gene or protein of interest and includes, but is not limited to, any composition or label detectable by spectroscopic, photochemical, electrical, optical or chemical means. Useful labels in the present invention include: biotin for staining with labeled streptavidin conjugate, magnetic beads (e.g., DYNABEADS™), fluorescent dyes (e.g., fluorescein, Texas red, rhodamine, green fluorescent protein, and the like), radiolabels (e.g., 3H, 125I, 35S, 14C, or 32P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

In addition, the means for detecting of the assay kit of the present invention can be immobilized on a substrate. Such a substrate can include any suitable substrate for immobilization of a detection reagent such as would be used in any of the previously described methods of detection. Briefly, a substrate suitable for immobilization of a means for detecting includes any solid support, such as any solid organic, biopolymer or inorganic support that can form a bond with the means for detecting without significantly affecting the activity and/or ability of the detection means to detect the desired target molecule. Exemplary organic solid supports include polymers such as polystyrene, nylon, phenol-formaldehyde resins, and acrylic copolymers (e.g., polyacrylamide). The kit can also include suitable reagents for the detection of the reagent and/or for the labeling of positive or negative controls, wash solutions, dilution buffers and the like. The kit can also include a set of written instructions for using the kit and interpreting the results.

The kit can also include a means for detecting a control marker that is characteristic of the cell type being sampled can generally be any type of reagent that can be used in a method of detecting the presence of a known marker (at the nucleic acid or protein level) in a sample, such as by a method for detecting the presence of a biomarker described previously herein. Specifically, the means is characterized in that it identifies a specific marker of the cell type being analyzed that positively identifies the cell type. For example, in a colon tumor assay, it is desirable to screen colon epithelial cells for the level of the biomarker expression and/or biological activity. Therefore, the means for detecting a control marker identifies a marker that is characteristic of an epithelial cell and preferably, a colon epithelial cell, so that the cell is distinguished from other cell types, such as a connective tissue or inflammatory cell. Such a means increases the accuracy and specificity of the assay of the present invention. Such a means for detecting a control marker include, but are not limited to: a probe that hybridizes under stringent hybridization conditions to a nucleic acid molecule encoding a protein marker; PCR primers which amplify such a nucleic acid molecule; an aptamer that specifically binds to a conformationally-distinct site on the target molecule; and/or an antibody, antigen binding fragment thereof, or antigen binding peptide that selectively binds to the control marker in the sample. Nucleic acid and amino acid sequences for many cell markers are known in the art and can be used to produce such reagents for detection.

The assay kits and methods of the present invention can be used not only to identify patients that are predicted to be responsive to a particular anticancer compound, but also to identify treatments that can improve the responsiveness of cancer cells which are resistant to anticancer compounds of the invention, and to develop adjuvant treatments that enhance the response of the anticancer compounds of the invention.

Each publication or patent cited herein is incorporated herein by reference in its entirety.

The invention now being generally described will be more readily understood by reference to the examples provided in Appendix I to this disclosure, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

Commercially available reagents referred to in these examples were used according to manufacturer's instructions unless otherwise indicated. The source of certain reagents is described below.

Constructs-Venus-tagged Parkin and mTurquoise-tagged Parkin S65A constructs were described previously (Zhang, C., et al., (2014), supra). PINK1-EGFP was constructed by inserting EGFP at carboxyl terminus of PINK1 in the background of CSII-EF-DEST-IRES-Hygromycin lentiviral vectors. pMSCV-CMV-puro-IMS-RFP was used as mitochondrial marker as described previously. LC3 and murine Bcl-2 were cloned into CSII-EF-CFP-DEST-IRESHygromycin lentiviral vectors to get CFP-LC3 and Bcl-2 constructs. Cell culture, transfection and reagent treatment—HEK293T cells were obtained from the ATCC (American Type Culture Collection). Parkin and PINK1 wild type and null MEF cells were described previously (Gautier, C. A., et al., (2008) Loss of PINK1 causes mitochondrial functional defects and increased sensitivity to oxidative stress. Proc Natl Acad Sci U S A. 105:11364-69). HeLa and HEK293T cells were maintained in Dulbecco's Modified Eagle Medium (DMEM) supplemented with 10% fetal calf serum (Invitrogen), penicillin, streptomycin (100 IU/ml and 100 mg/ml, respectively), and L-Glutamine. MEFs were grown in Dulbecco's Modified Eagle Medium containing 10% fetal bovine serum, penicillin (100 U/mL), streptomycin (100 U/mL), 1 mM I-glutamine, 1 mM Na-pyruvate, and 1×nonessential amino acids. All the stable cell lines made by lentivirus and retrovirus packaging were selected with 100 μg/ml hygromycin (Alexis Biochemicals), 5 μg/ml blasticidin (Invitrogen), or 2 μg/ml puromycin (Sigma) based on the selection markers. Mitochondrial membrane potential was dissipated with 10-20 μM carbonyl cyanide m-chlorophenyl hydrazone (CCCP, Sigma-Aldrich) and 10-40 μM sorafenib and sorafenib tosylate as indicated (Selleckchem and LC Laboratories). For apoptosis and mitophagy assays, cells were treated with 200 nM MitoTracker red dye (Life Technologies), 20 μM Z-VAD-FMK (Abcam), 4 μM Obatoclax (LC Laboratories) and 10 μM ABT-737 (AdooQ). Antibodies-Antibodies used in this study for western blot (WB) were: mouse anti-Parkin (1:1000 and 1:5000; clone PRK8, Sigma-Aldrich), rabbit anti-PINK1 (1:1000, BC100-494, Novus Biologicals; 1:1000, D8G3, Cell Signaling), mouse anti-EZRIN (1:5000, Sigma-Aldrich) and mouse anti-GAPDH(1:5000, Santa Cruz). Anti-caspase-3, PARP, Mcl-1, Bcl2, Bcl-xL antibodies were purchased from Cell Signaling Technology. Live cell imaging and fluorescence microscopy: To obtain high throughput images and movies, cells were grown on Costar 96-well 3603 plates. Molecular Devices ImageXpress XL was used to screen the plates and collect data. Live cell imaging was collected for 0-24 hours. To get high resolution images and movies for Parkin mitophagy, cells were grown on a 4 well glass bottom chamber (LabTek). Confocal images were acquired on a Nikon A1R Confocal and TIRF using a 40× or 100× (NA 1.45) objective lens. Mitochondrial membrane potential quantification-Mitochondria in HeLa cells were stained using 400 nM tetramethylrhodamine, methyl ester (TMRE) (Life Technologies). The mitochondrial membrane potential was measured using the mCherry channel on ImageXpress microscope (Molecular Devices). The 20× objective was used, and images were collected every two minutes for 40 minutes. At least 500 cells per condition were examined and quantified. Electron transport chain activity assays-Confocal laser scanning live cell imaging of FAD autofluorescence was used to estimate the activity of the ETC on an A1R laser scanning confocal microscope (Nikon) following the protocol described in (Bartolomé, F., and Abramov, A. Y. (2015) Measurement of mitochondrial NADH and FAD autofluorescence in live cells. Mitochondrial Medicine, 1:263-270). Cells growing on a glass bottom 96 well plate were scanned for 2 min with 454 nm excitation wavelength and emission between 505 and 550 nm was collected to establish the baseline autofluoresence. The laser power was limited to less than 0.2% to prevent cell damage. With the optimized scanning conditions and stabilized autofluoresence signal over 2 min, 1 μM of CCCP is added to maximize the respiration and continue scan for an additional 2 min. To evaluation to effect of sorafenib on FAD autofluroscence, cells were pretreated with either 20 μM sorafenib or 10 μM antimycin A for 2 min prior to the addition of 10 μM of CCCP. Mitochondrial ETC and complex V assays-MitoTox™ complex I, complex IV and complex V OXPHOS activity microplate assay kits were purchased from Abcam (ab109903 & ab110419) and used to measure the inhibitory activity of compounds on Complex I, IV and V, respectively following the manufacturer's instruction. Mitocheck complex II/III (Cayman Chemical Cat no. 700950) assay kit was used to measure the inhibitory activity of compounds on complex II/III and IV respectively. Briefly, for complex I, IV and V activity measurement, bovine heart mitochondria (360 μl at 5.5 mg/ml) was solubilized, 15 μg of mitochondria was added to each well of precoated 96 well microplate. NADH ubiquinone oxidoreductase, Cytochrome c oxidase, ATP synthase were immunocaptured by the corresponding antibodies coated on 96 well assay plates. For complex I assay, phospholipids were added prior to the incubation with 10 μM DMSO or 10 μM rotenone or different doses of sorafenib along with the complex I activity solution which contains Ubiquinone 1 and NADH. NADH depletion was calculated from decreasing absorbance at 340 nm at room temperature in kinetic mode taking absorbance measurements every minute for 1 hour. The rate of decrease in absorbance at 340 nm over time is calculated from the linear range using MATLAB for various experimental conditions. For complex IV assay, antibody coated microplate was washed with blocking buffer prior to the addition of 200 ng detergent-solubilized bovine heart mitochondria per well and 3 hr incubation at room temperature. 200 μl of complex IV activity solution which contains reduced cytochrome c along with 10 μM DMSO or 10 μM KCN and different dose of sorafenib was incubated at room temperature for 1 hr. Cytochrome c oxidation was measured by taking the absorbance at 550 nm in kinetic mode every minute for 60 minutes. Since the complex IV reaction is product inhibited, the rate of activity was expressed as the initial rate of oxidation of cytochrome c. For complex V assay, 15 μg of detergent-solubilized bovine heart mitochondria was added to each well of microplate precoated with the complex V antibody. After 2 hr incubation and wash, 40 μl of phospholipids was added and incubated for 45 min prior to the addition of the complex V activity solution which contains Phosphoenolpyruvate (PEP), Pyruvate kinase (PK), lactate dehydrogenase (LDH) and NADH. The production of ADP by ATP synthase can be coupled to the oxidation of NADH to NAD+, and the progress of the coupled reaction monitored as a decrease in absorbance at 340 nm. The complex V activity solution was added to microplate wells along with 10 μM DMSO or 10 μM oligomycin or different dose of sorafenib. The activity of the ATP synthase enzyme is measured by taking absorbance at OD 340 nm every minutes for 1 hour at room temperature and the rate is calculated as the linear rate of activity between 12 and 50 minutes. For complex II/III assay, 20 μl of intact bovine heart mitochondria was pre-incubated with 2 μM rotenone and 20 μM KCN prior to the addition of complex III activity assay buffer which contains succinate and oxidized cytochrome c. The rate of reduced cytochrome c production was measured by taking absorbance at OD 550 nm every minutes for 10 min at room temperature in the presence of 10 μM DMSO, 10 μM antimycin A, and different dose of Sorafenib. All data was collected with Tecan Safire reader and custom MATLAB scripts were used to analyze and plot the results. Each experiment was run in triplicate and repeated at least twice. Mitophagy assays-Mitophagy was determined by the colocalization study of LC3 with mitochondrial marker (RFP-Smac). The CFP-LC3 and RFP-Smac colocalization was quantified using custom MATALB scripts to calculate Pearson and Manders colocalization coefficients. Cell death assays-Four independent cell death assays were used. For the HeLa cells stably expressing RFP-Smac, cell death was monitored by quantifying RFP-Smac release from mitochondria as described previously (Spencer, S. L., et al., (2009) Non-genetic origins of cell-to-cell variability in TRAIL-induced apoptosis. Nature. 459, 428-32). Live cell imaging of cell apoptosis in MEF and HEK293 cells were performed using NucView™488 (Biotium). The detailed method was described previously (Zhang, C., et al., (2014) PINK1 triggers autocatalytic activation of parkin to specify cell fate decisions. Curr. Biol. 24:1854-65). Cells were grown on 96-well plates overnight, and then 4 μM NucView caspase-3 biosensor (Biotium) was added. HCS microscope ImageXpress (Molecular Devices) was employed to collect images at the indicated time. At least 1000 cells per well were examined and quantified. Hoechst dye was included in the media to obtain the total number of nuclei in the field of view. The number of NucView™488 positive cells relative to the total number of nuclei was determined and plotted. Apoptotic cell death was also visualized and scored by staining with Hoechst 33258 which labels all cells regardless cell health. The average intensity of DNA labeling significantly increased in apoptotic cells as nuclei condense during apoptosis. Quantitation of cell death was performed using automated high content analysis cell heath application module in MetaXpress software (Molecular Devices). Statistical analysis—The mitochondrial colocalization with Parkin and LC3 in HeLa cells and was assessed by visually scoring for colocalization coefficient more than 200 cells per stable cell line in at least three independent experiments. Images collected from a confocal microscope were used to get high resolution images. For quantification of HeLa apoptosis, more than 200 cells were quantified per condition by counting RFP-Smac releasing and chromosome condensing. For quantification of MEF and 293 apoptosis, more than 2000 cells were quantified per condition using MetaXpress application module Multiwavelength Cell Scoring Application Module (Molecular Devices). For quantification of high throughput scoring of Parkin and Mitochondia colocalization, more than 500 cells were quantified per condition using MetaXpress TransfluorColocalization Application Module (Molecular Devices). Standard deviations were calculated from at least three sets of data. The p values were determined using Microsoft Excel.

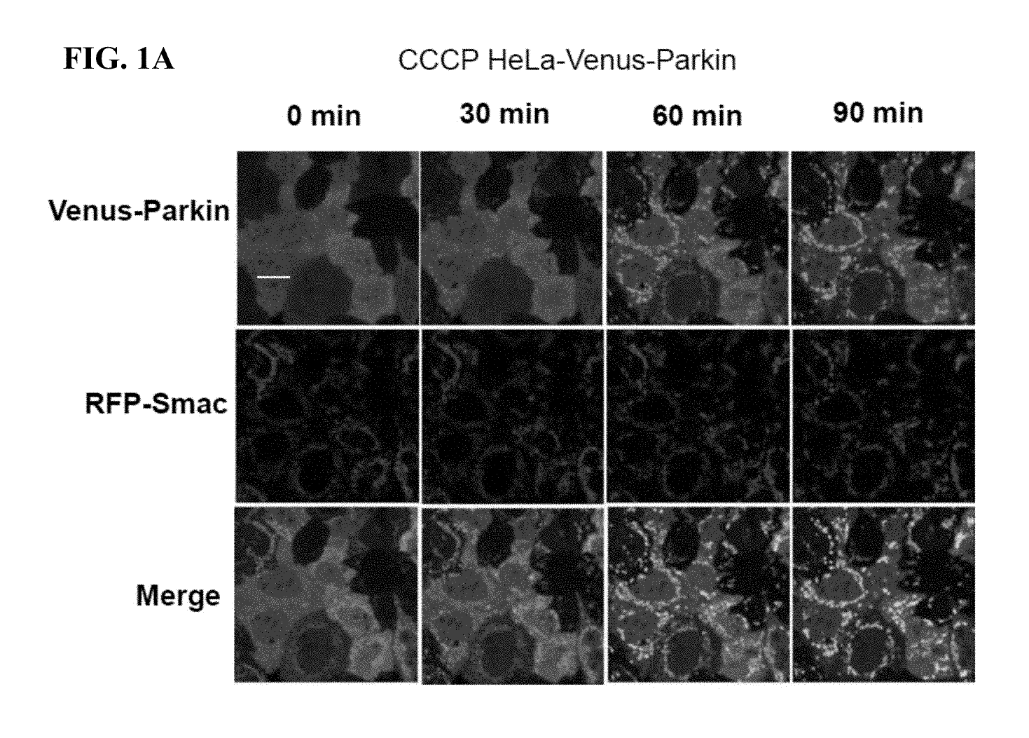

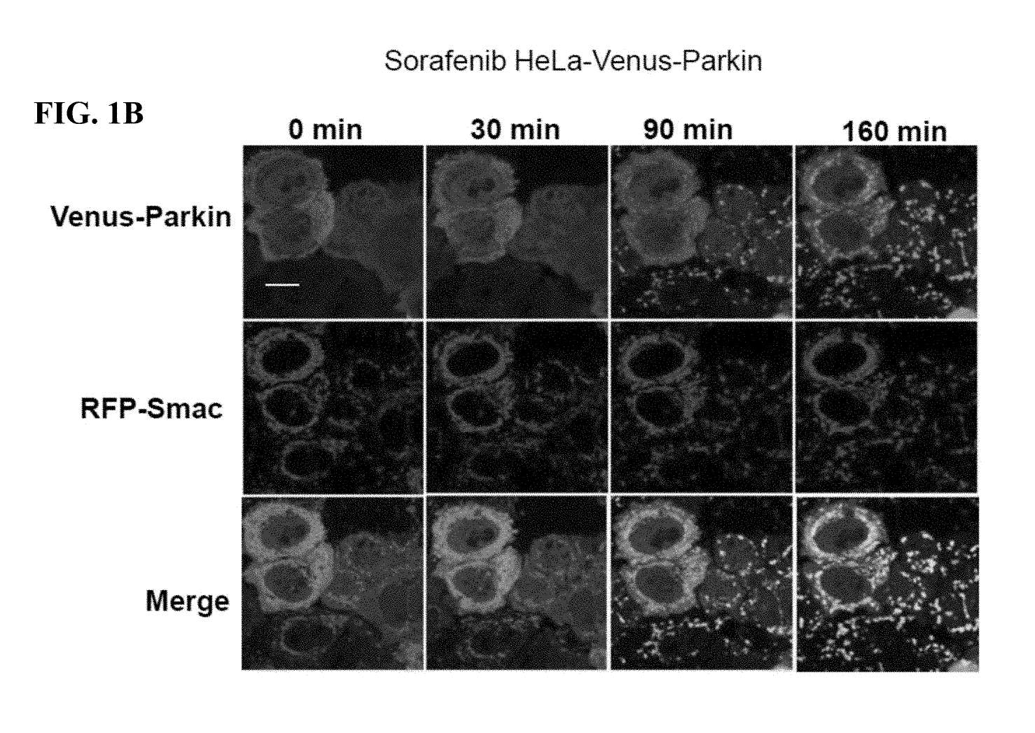

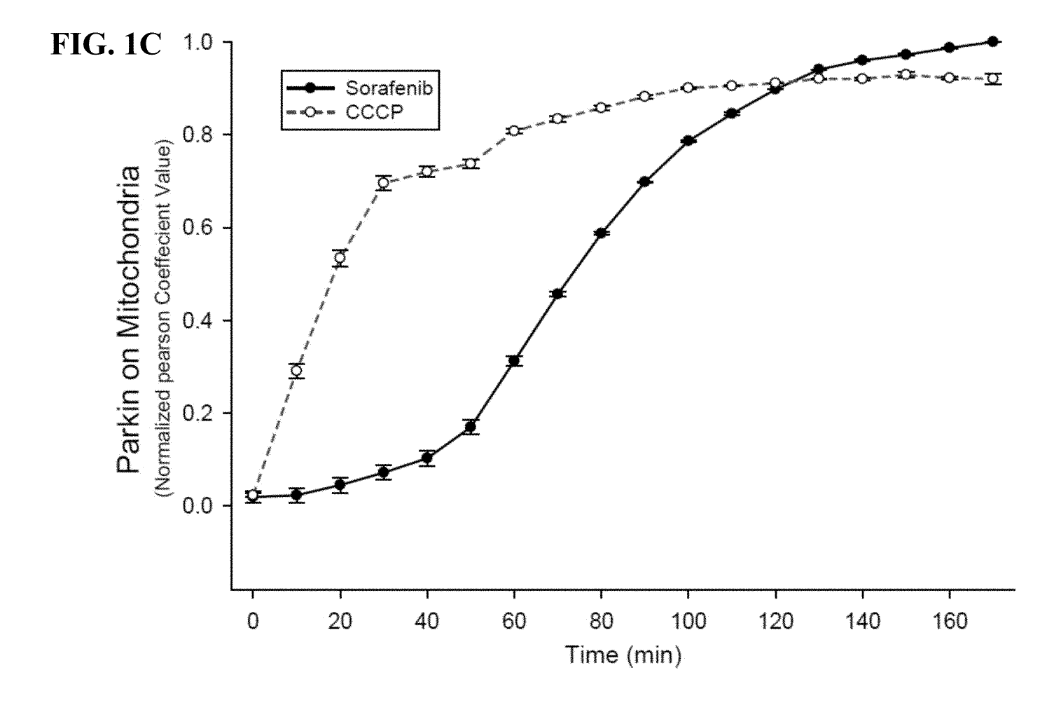

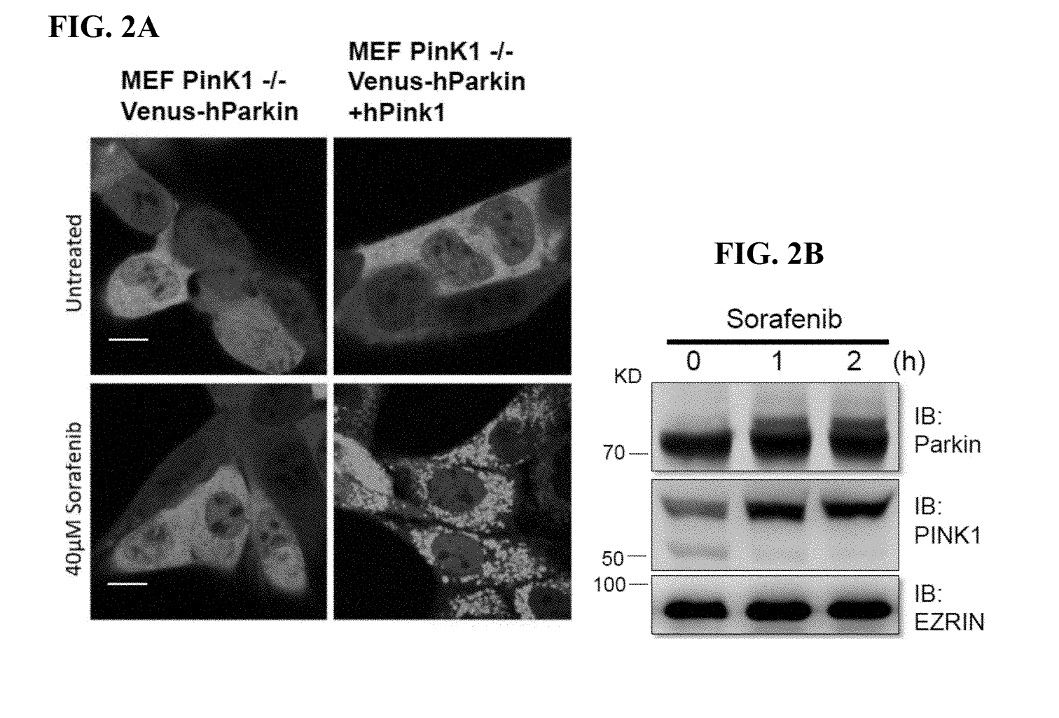

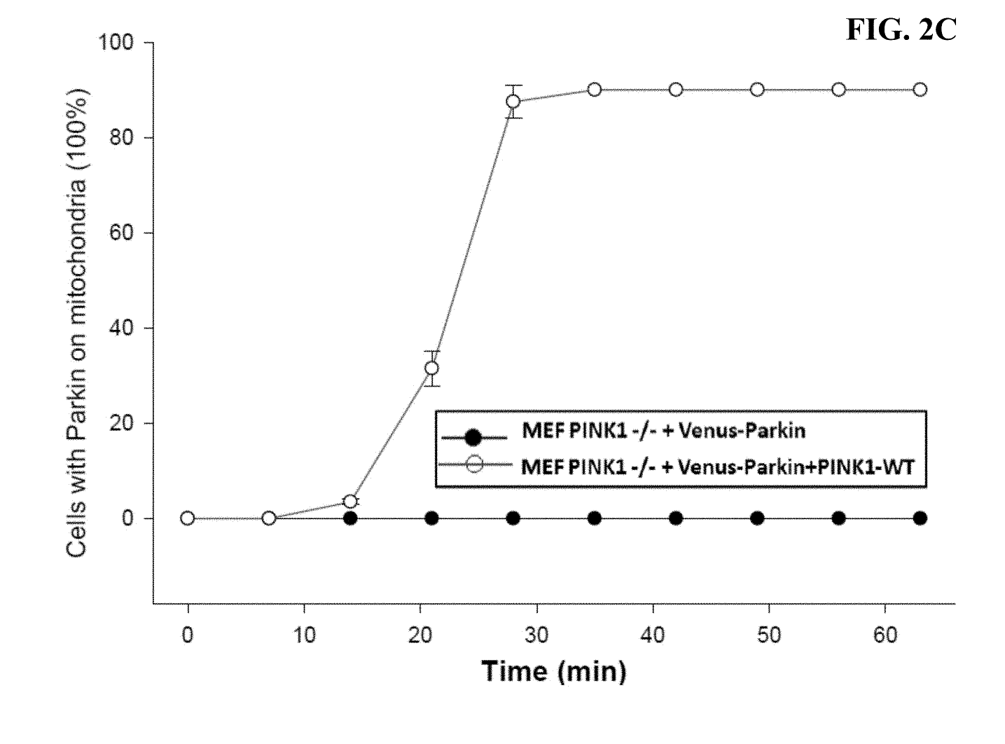

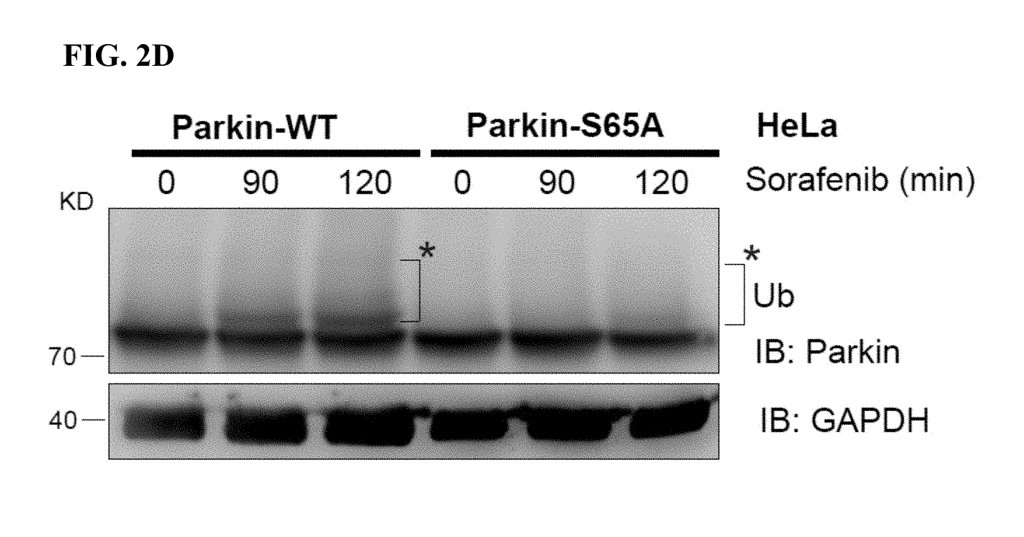

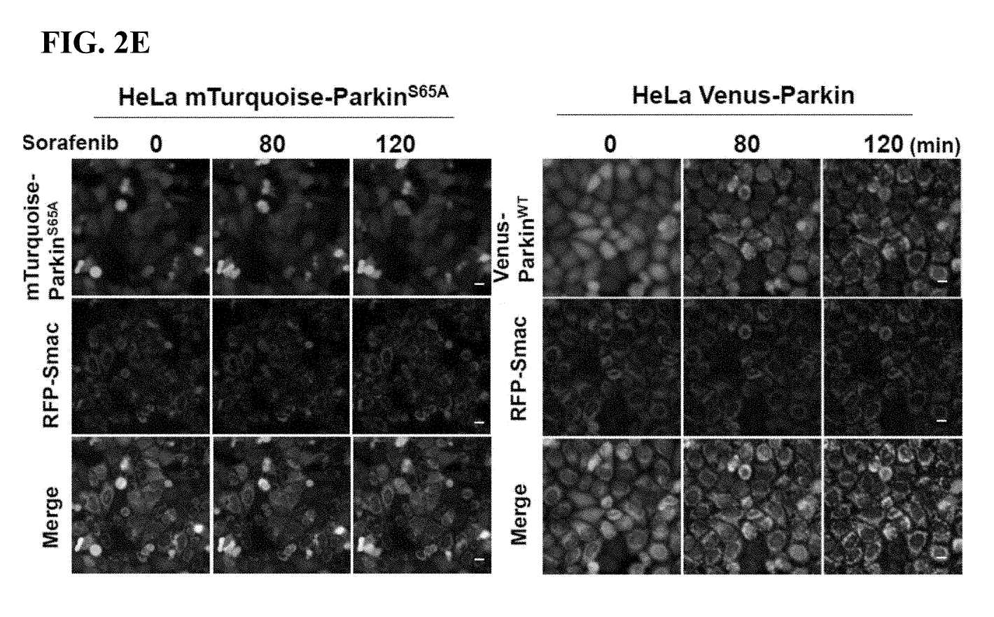

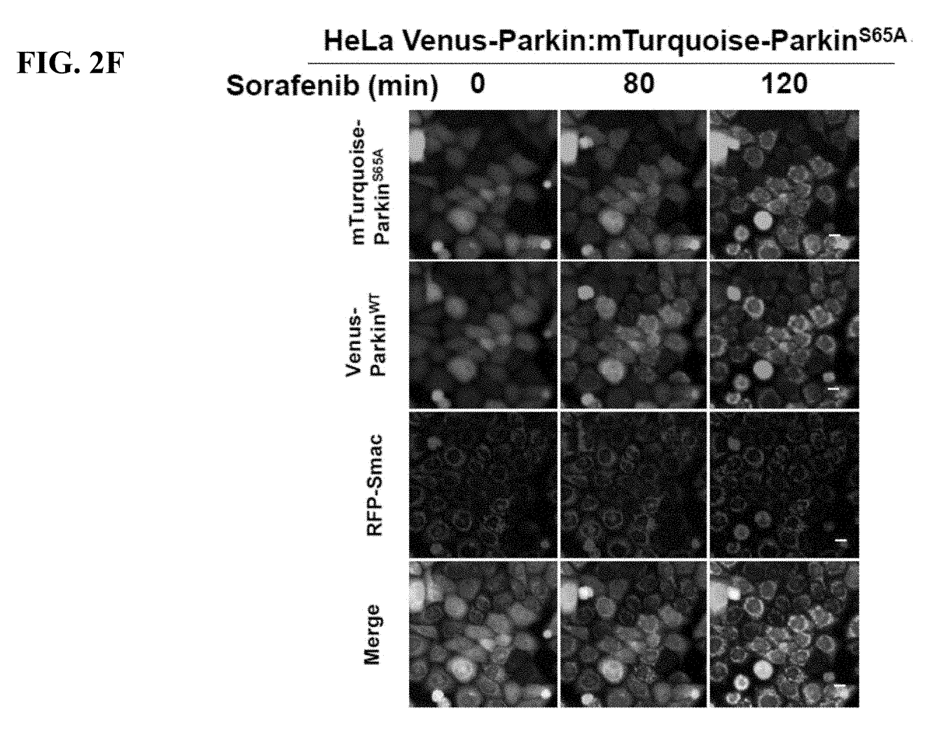

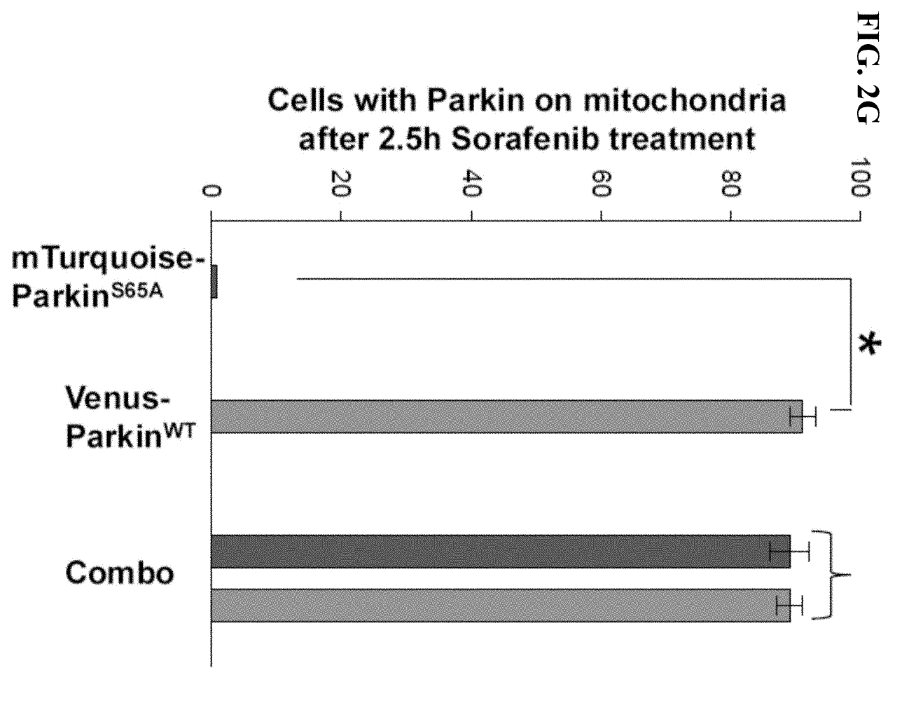

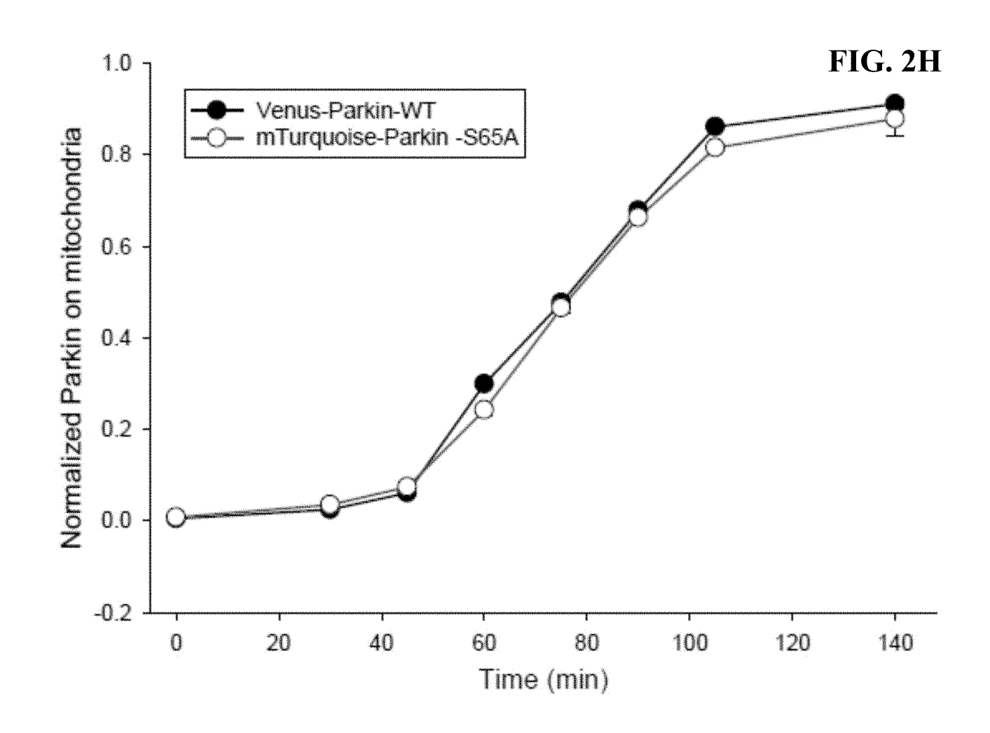

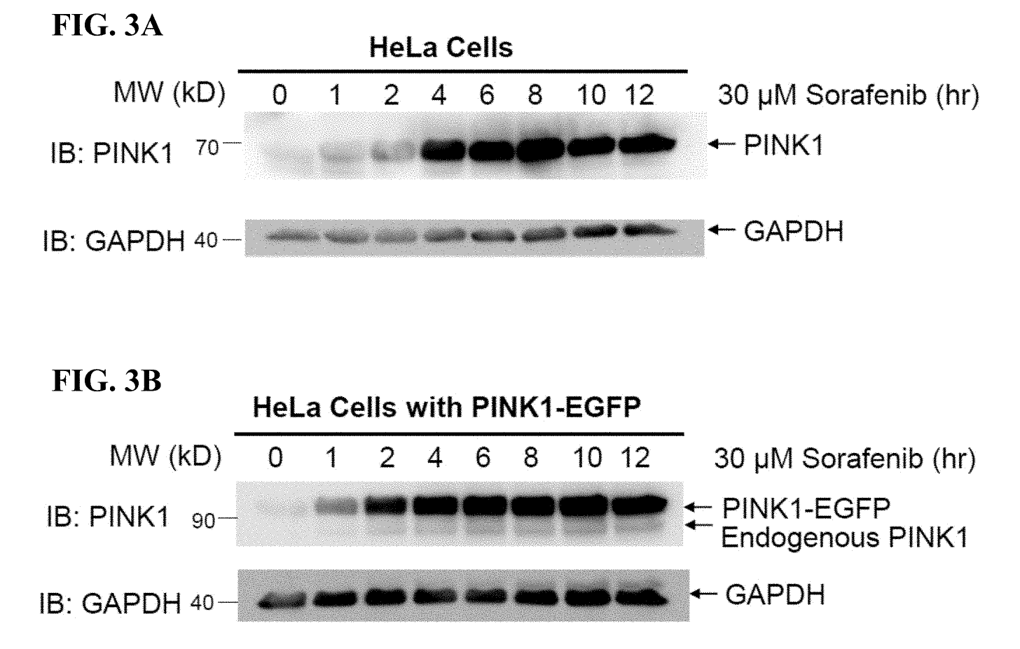

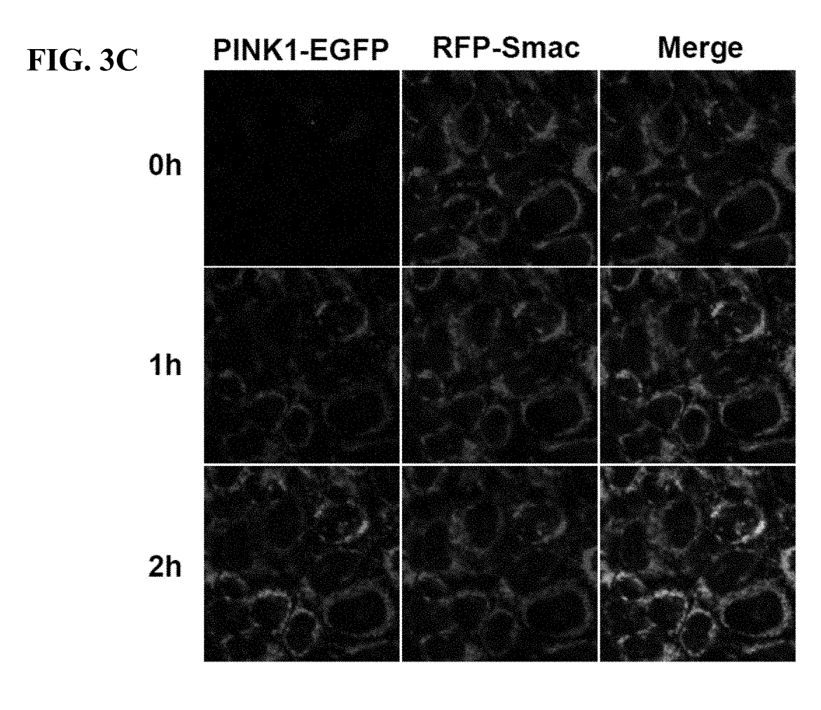

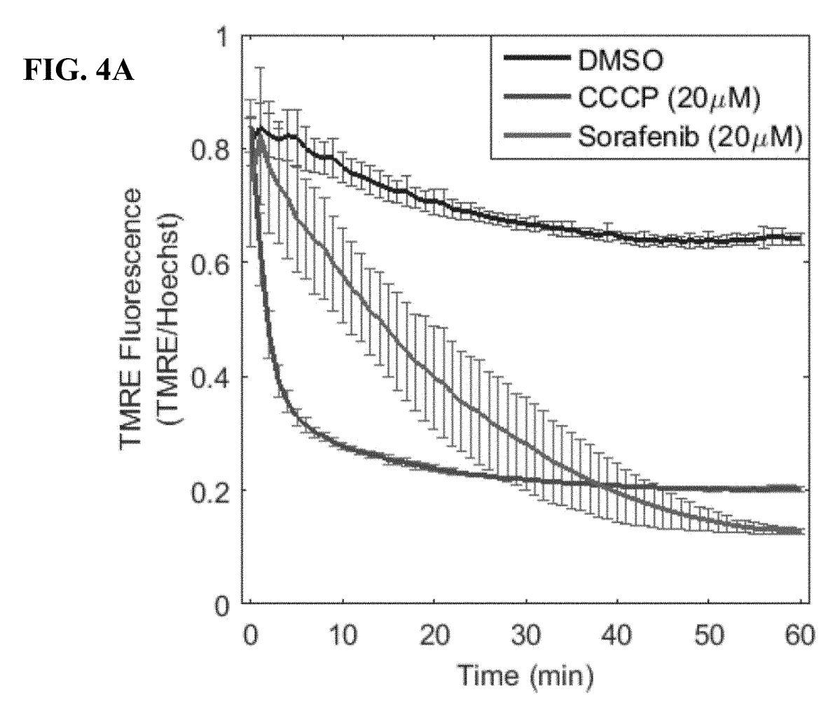

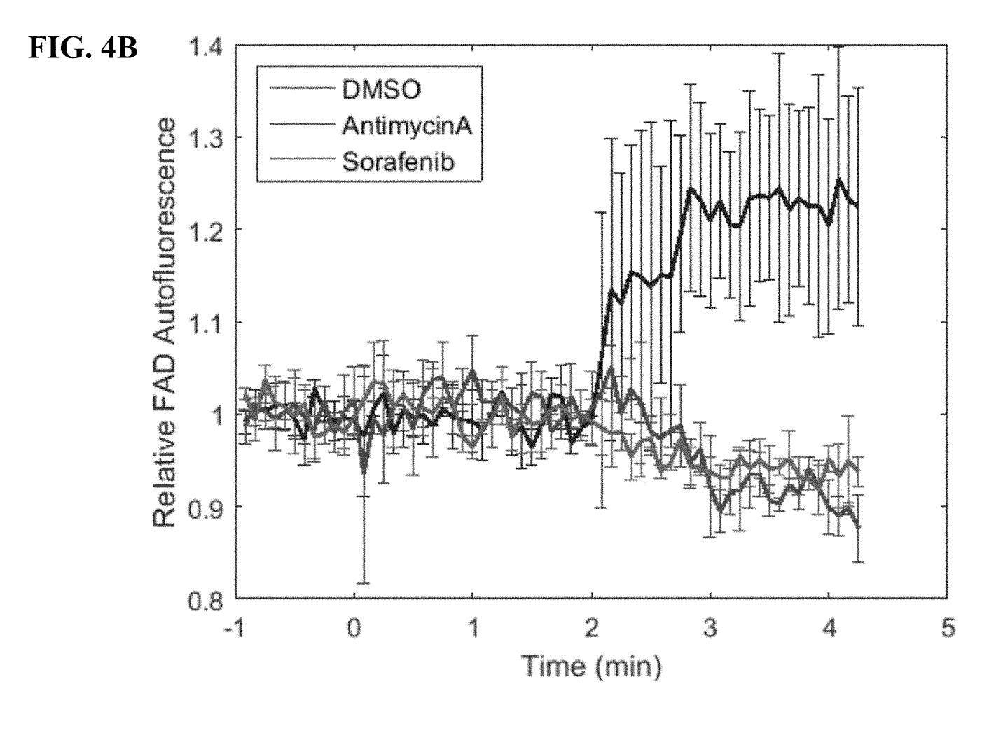

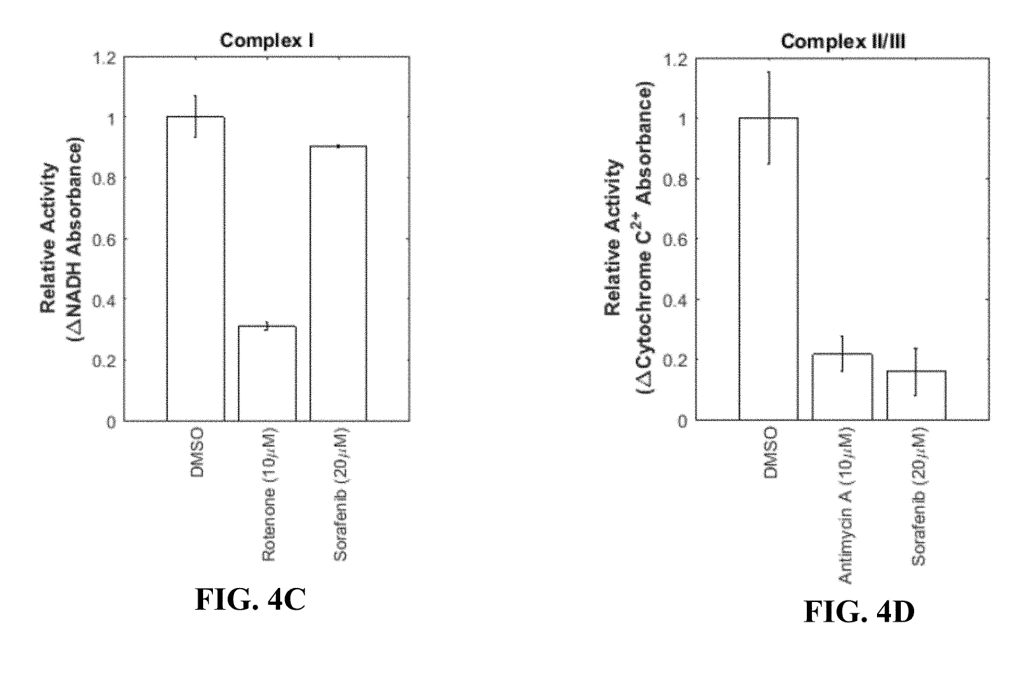

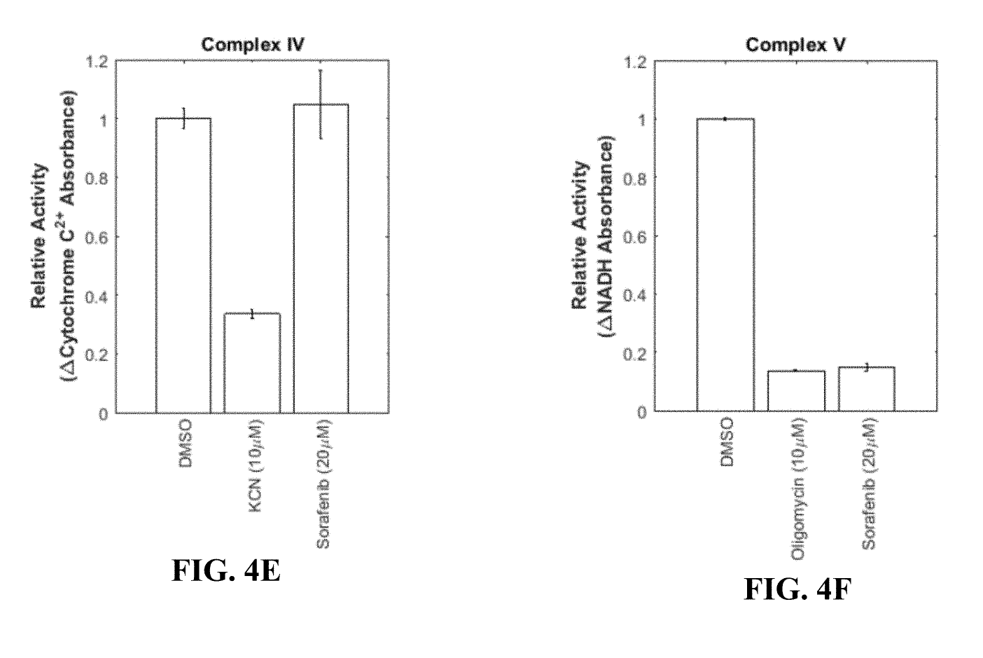

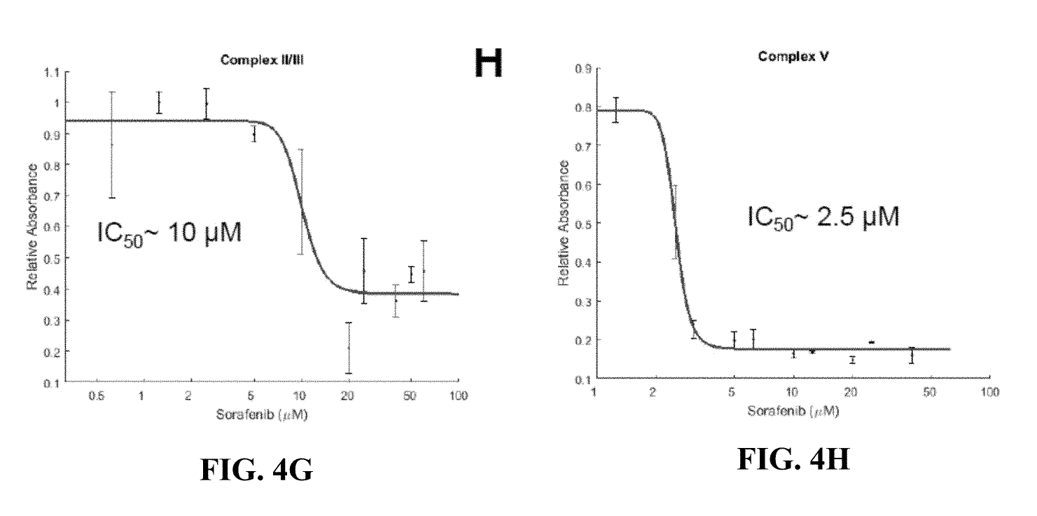

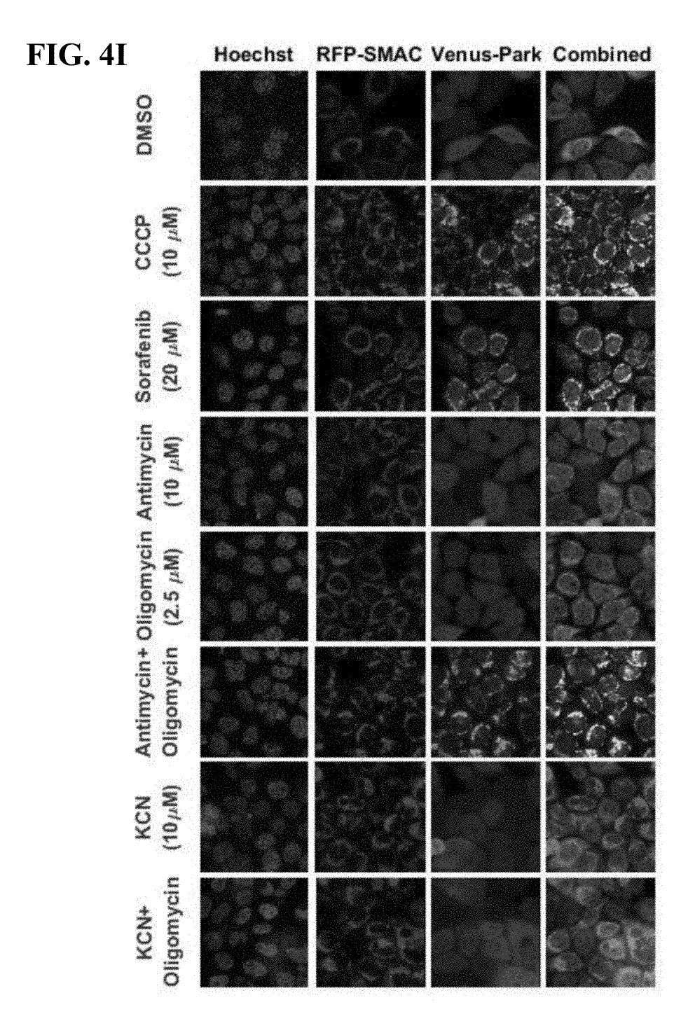

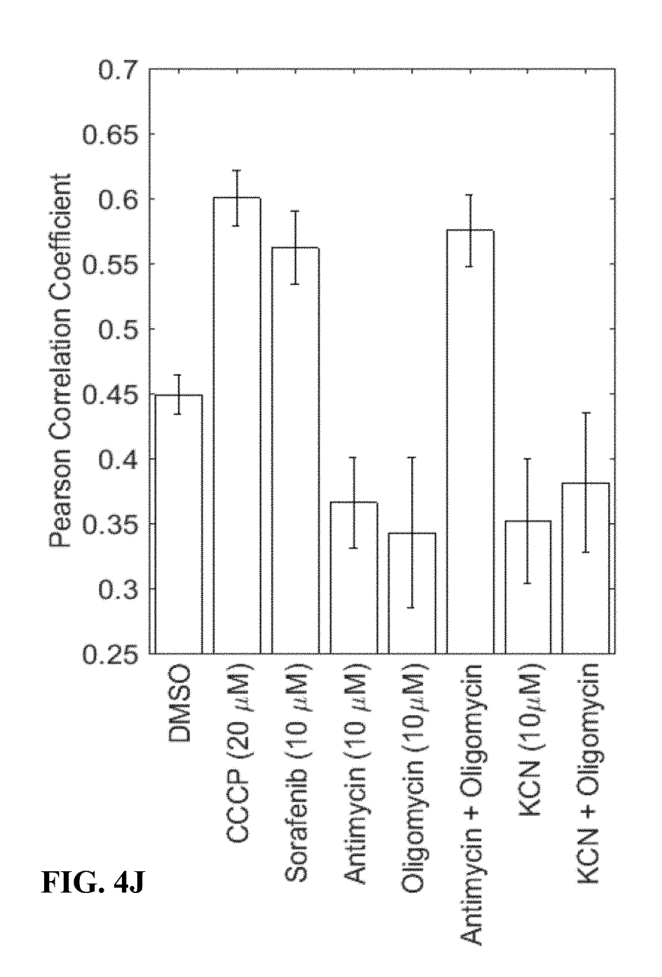

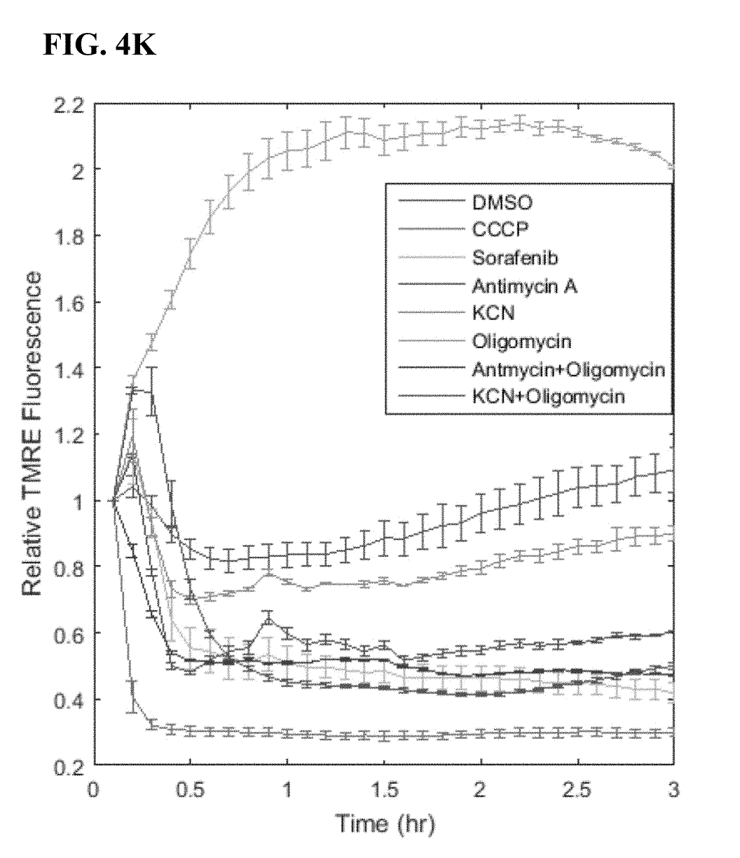

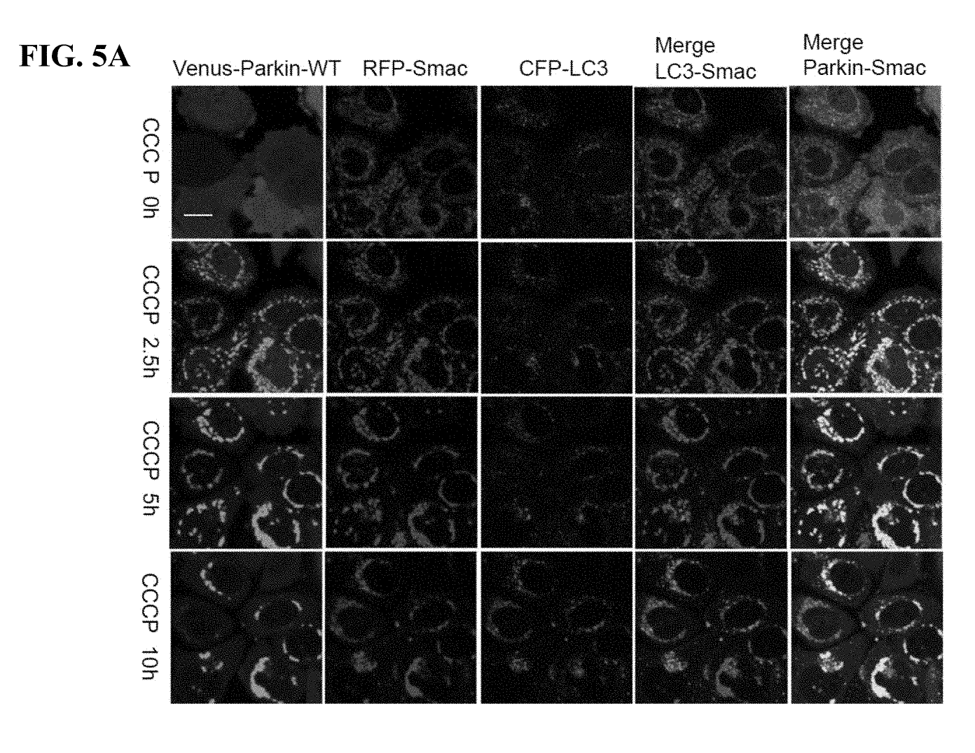

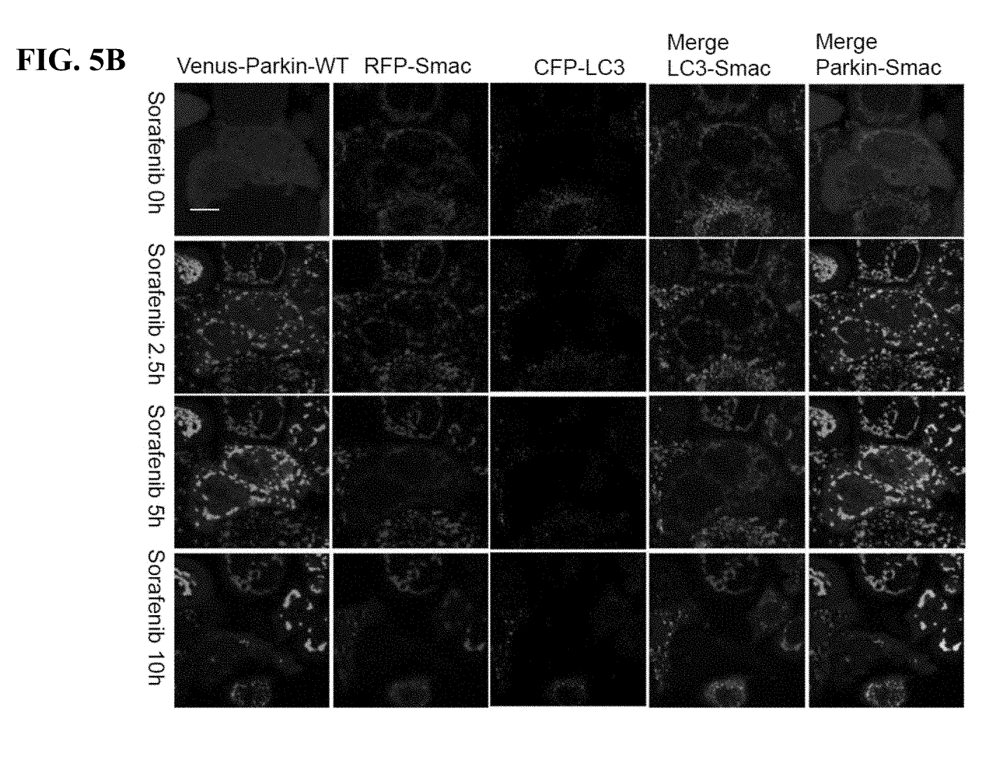

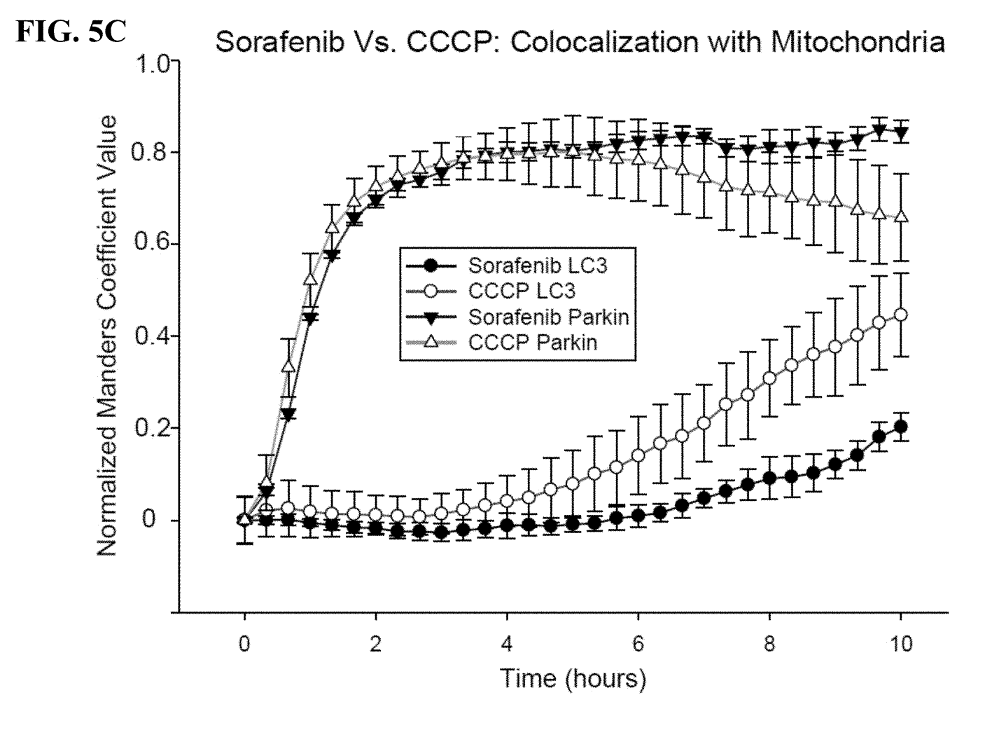

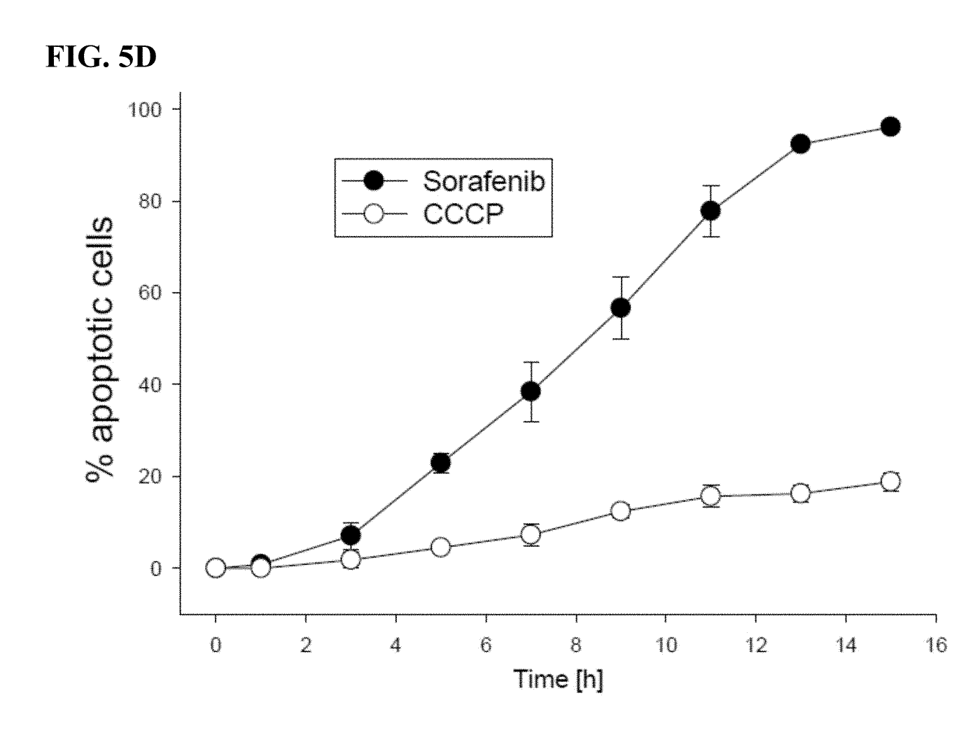

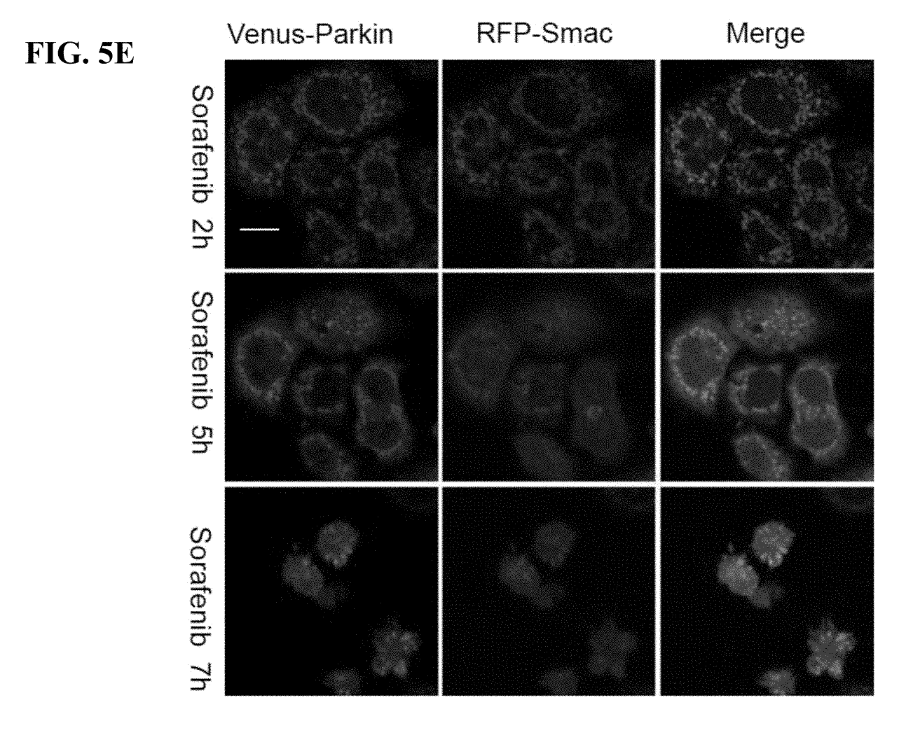

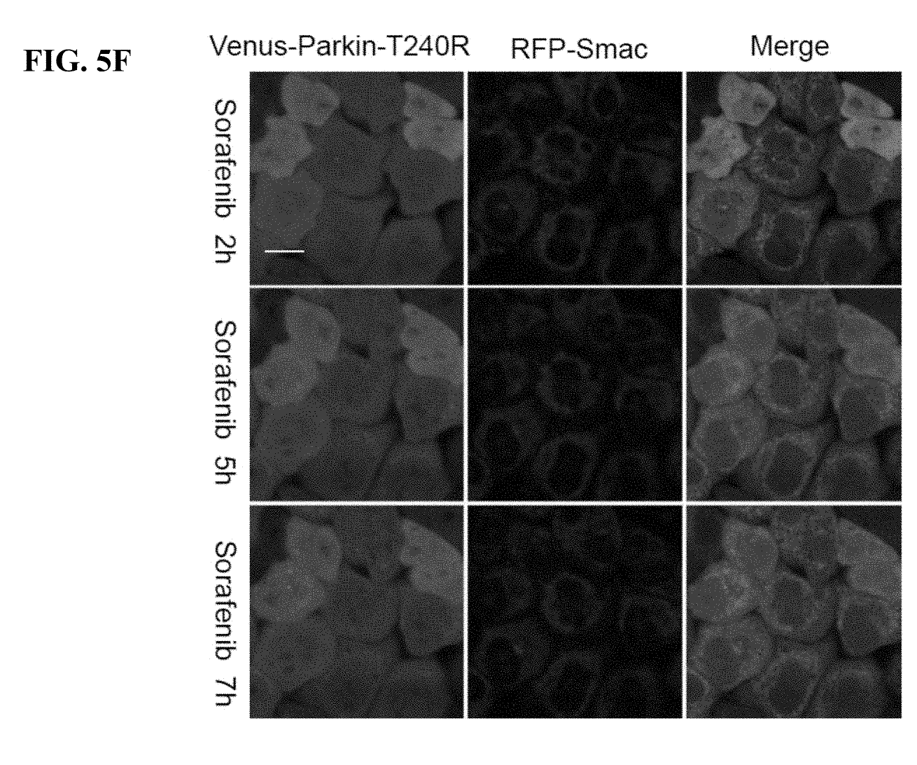

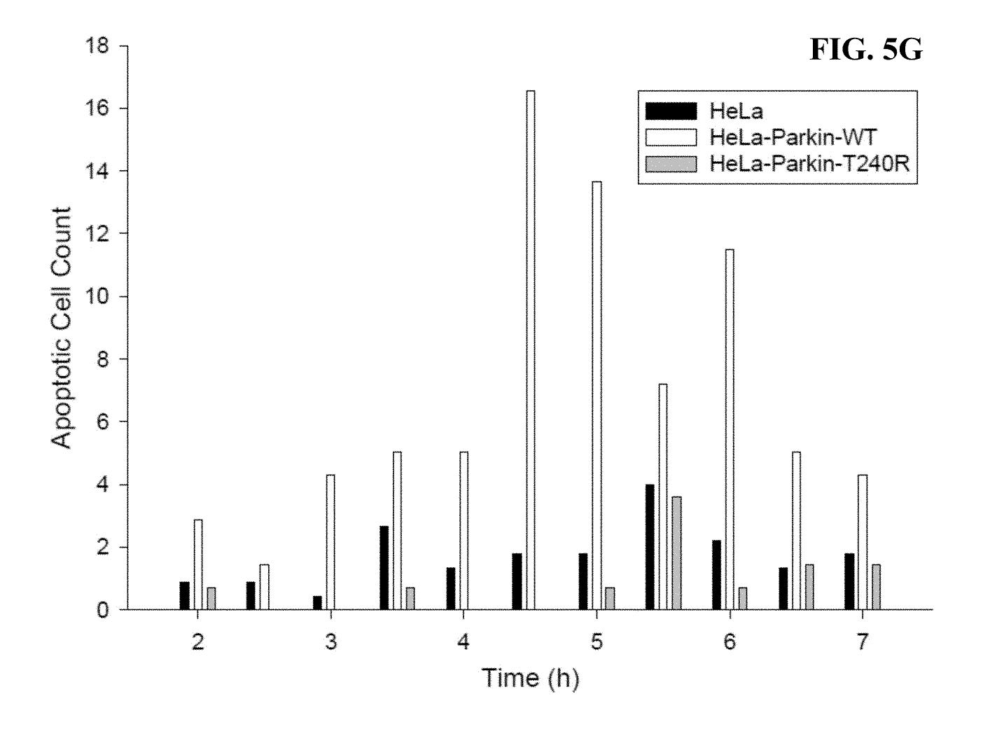

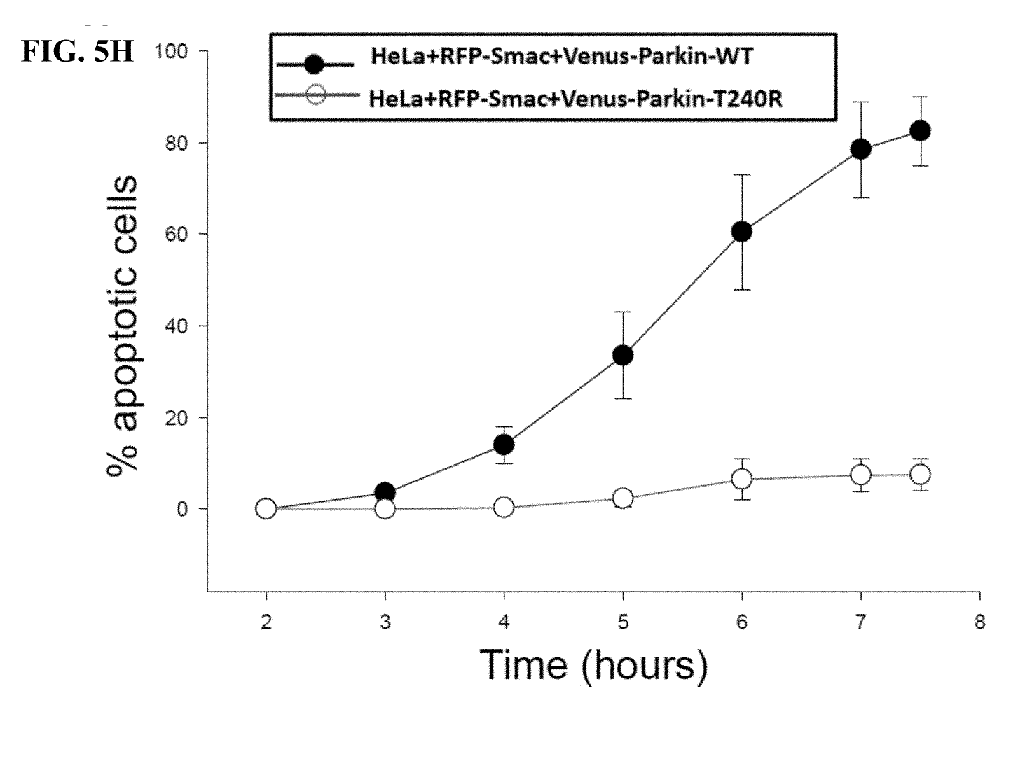

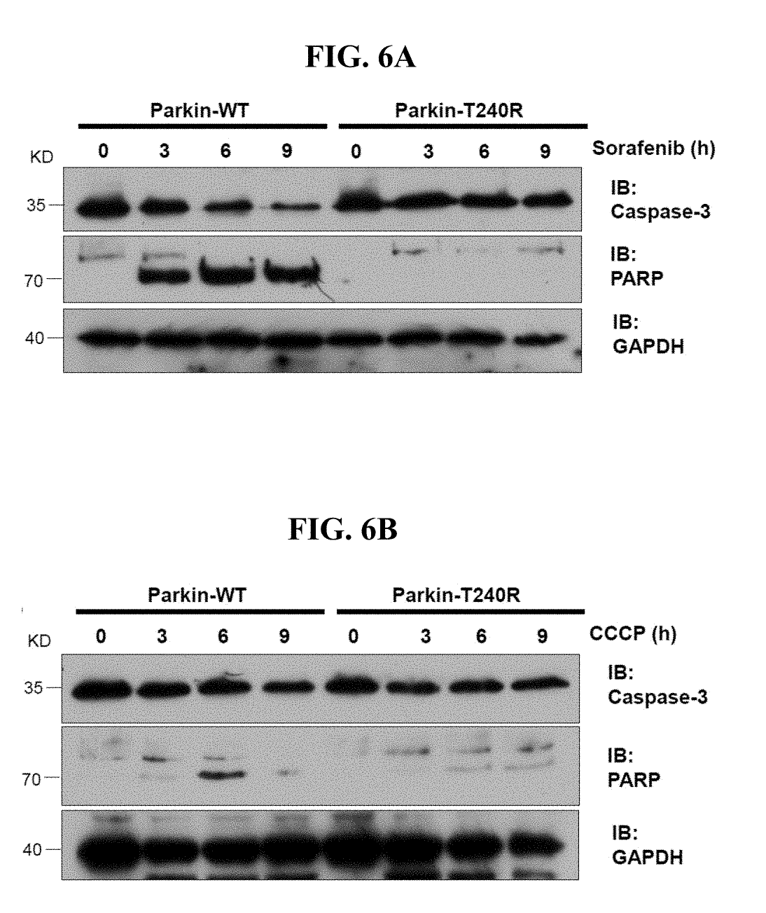

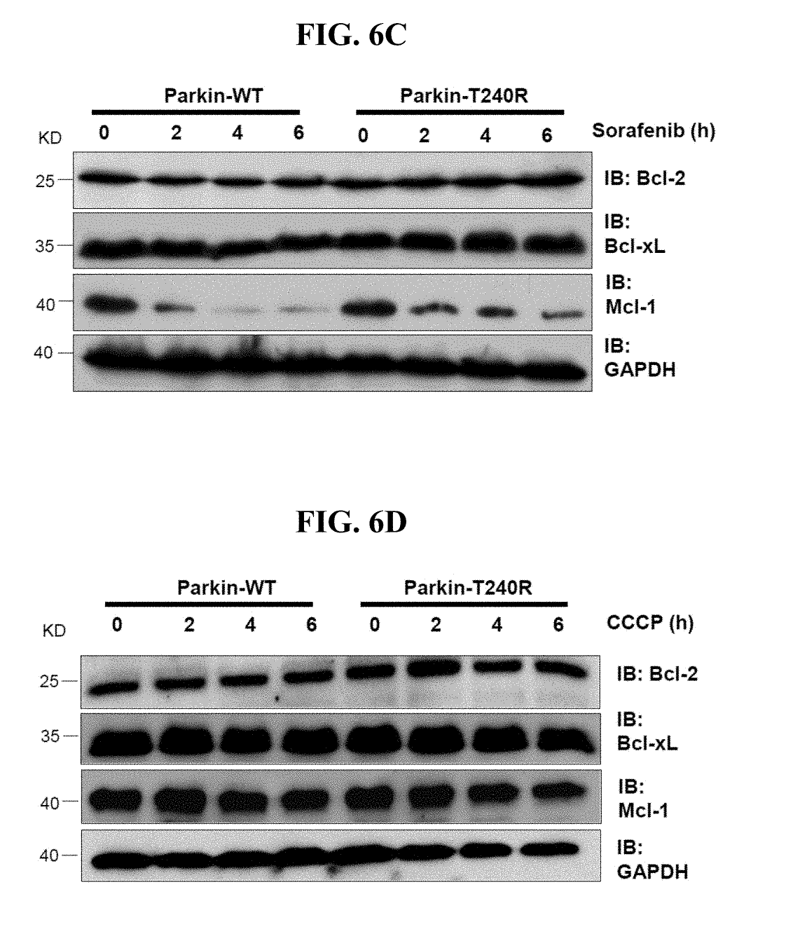

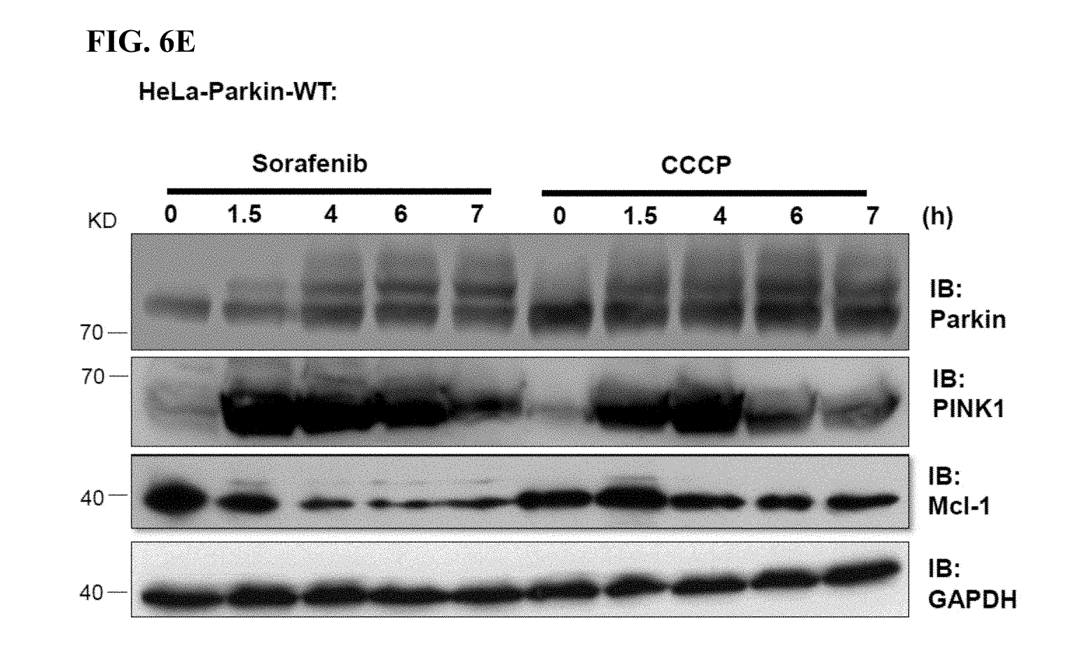

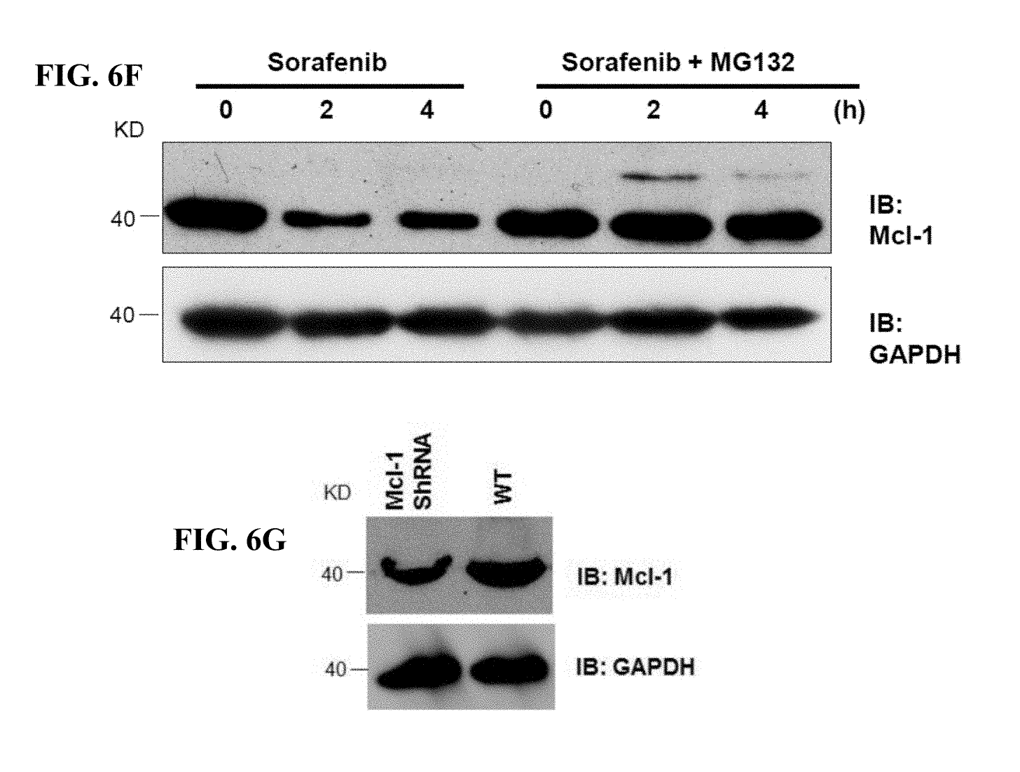

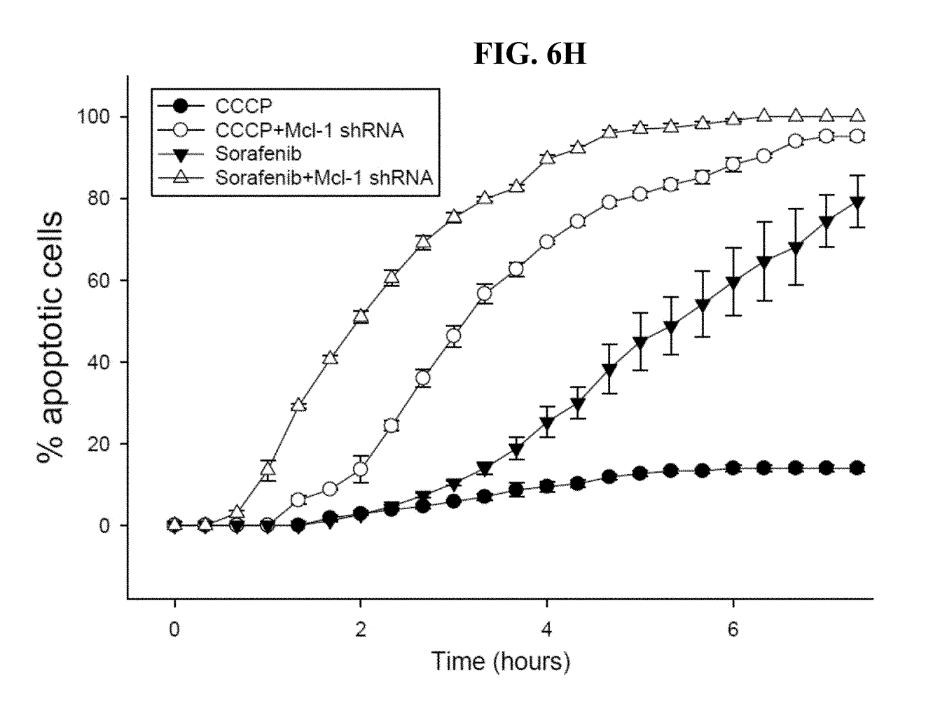

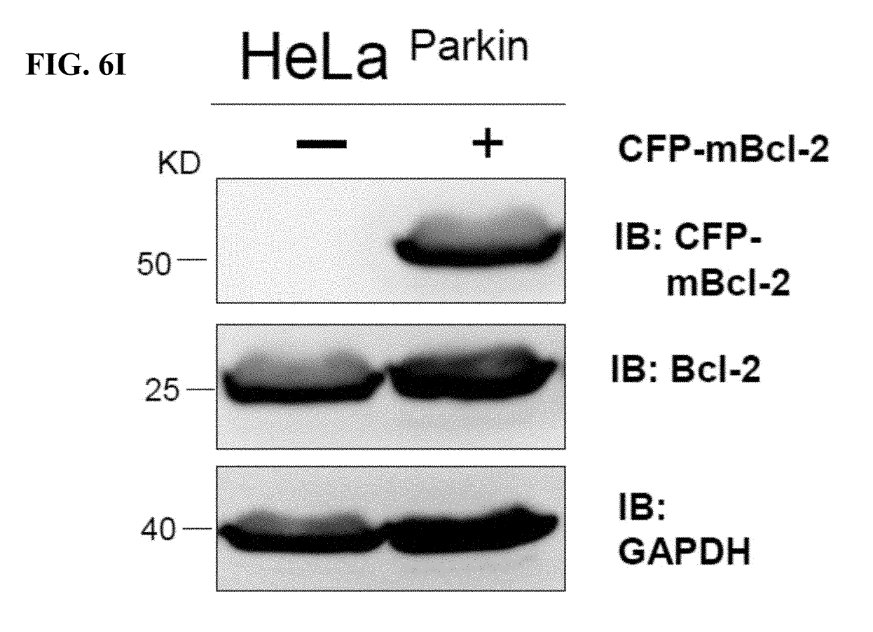

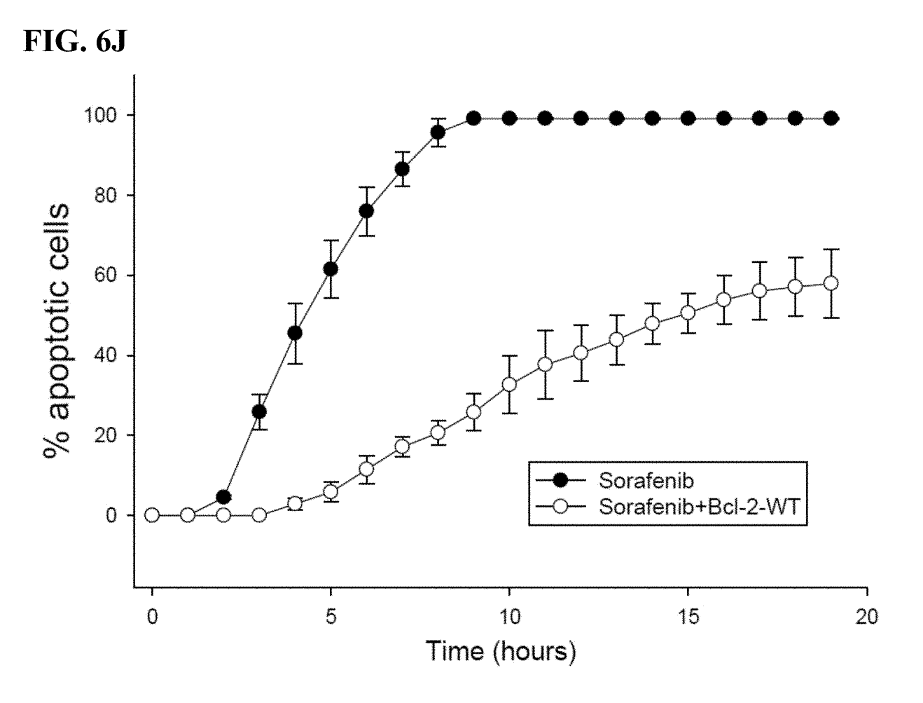

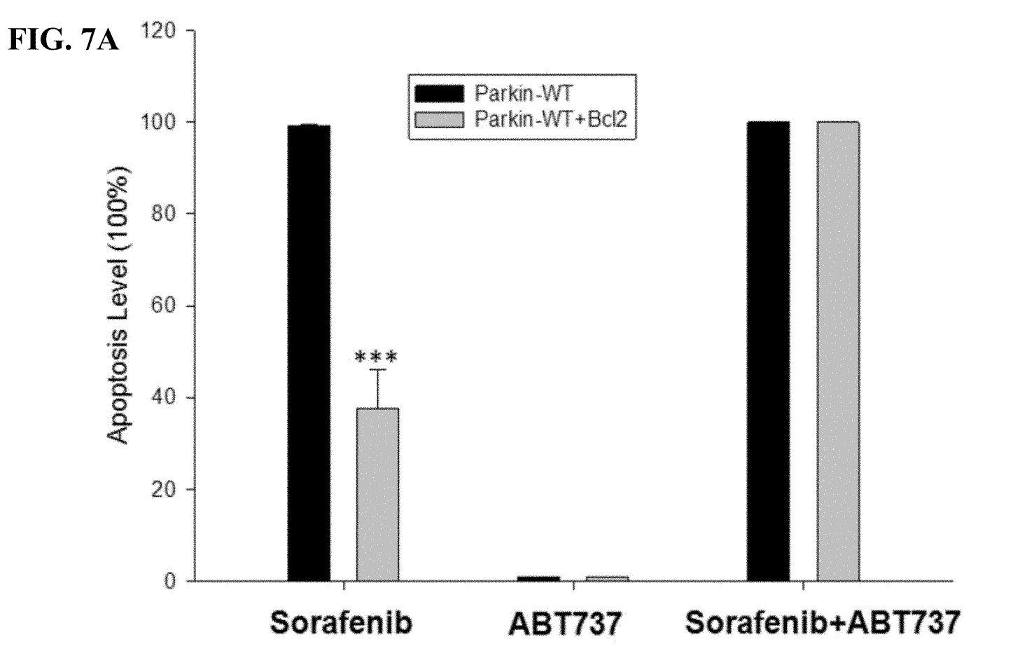

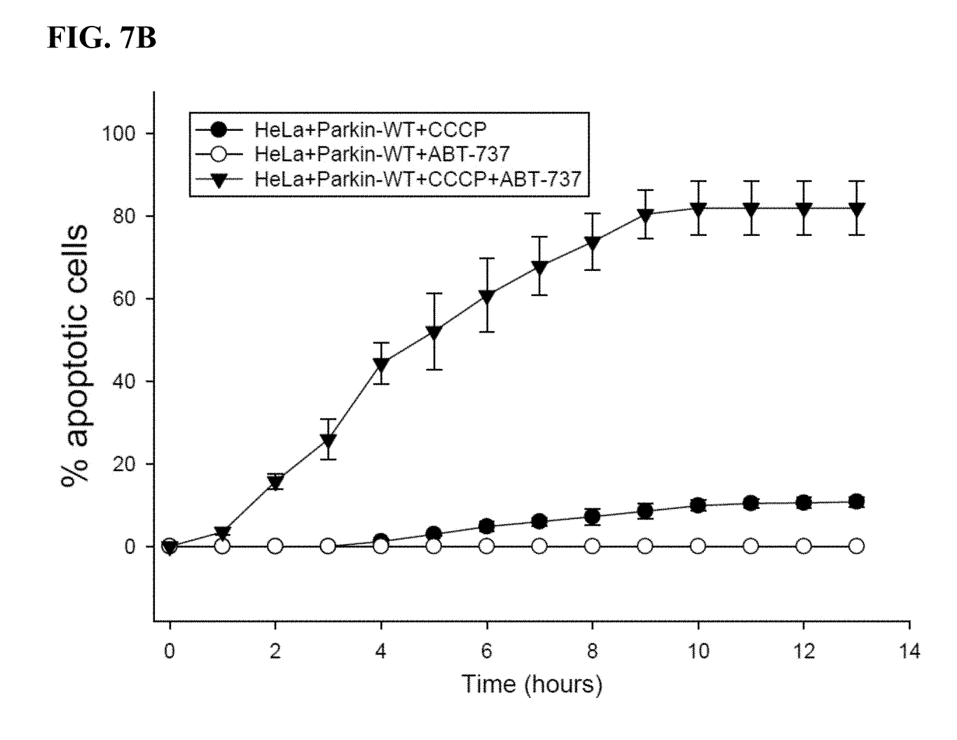

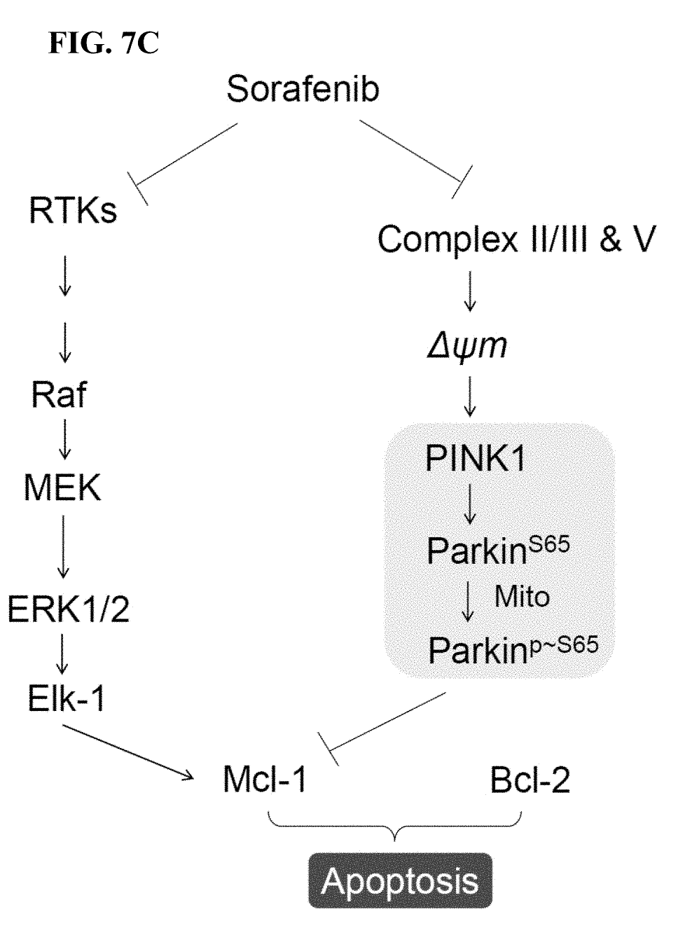

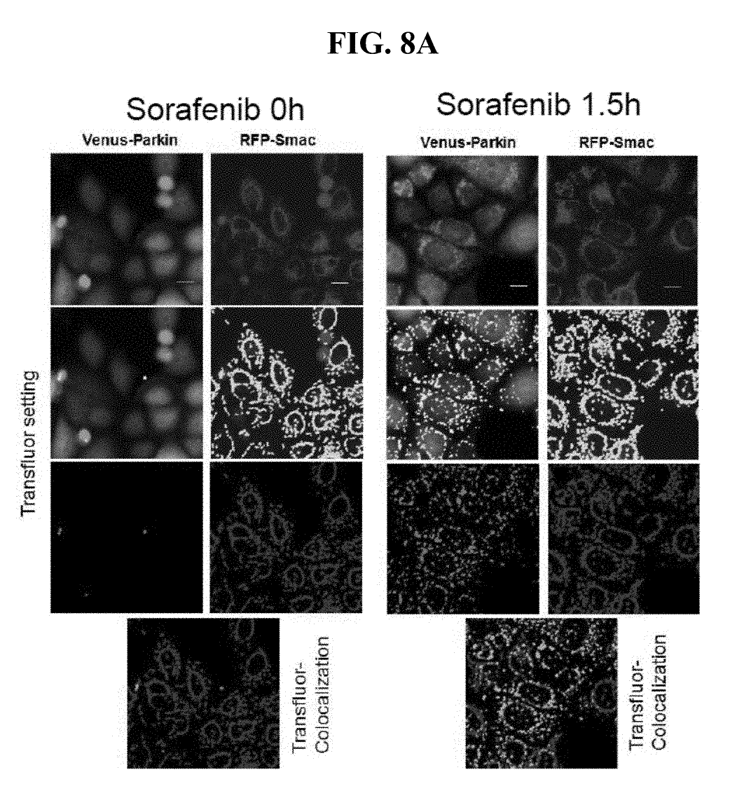

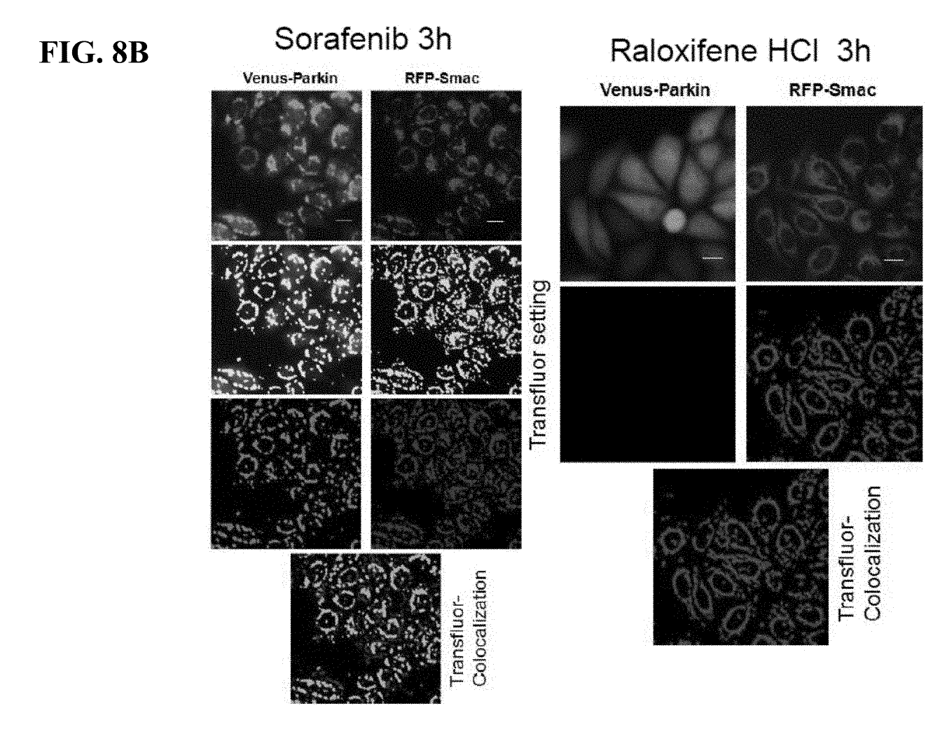

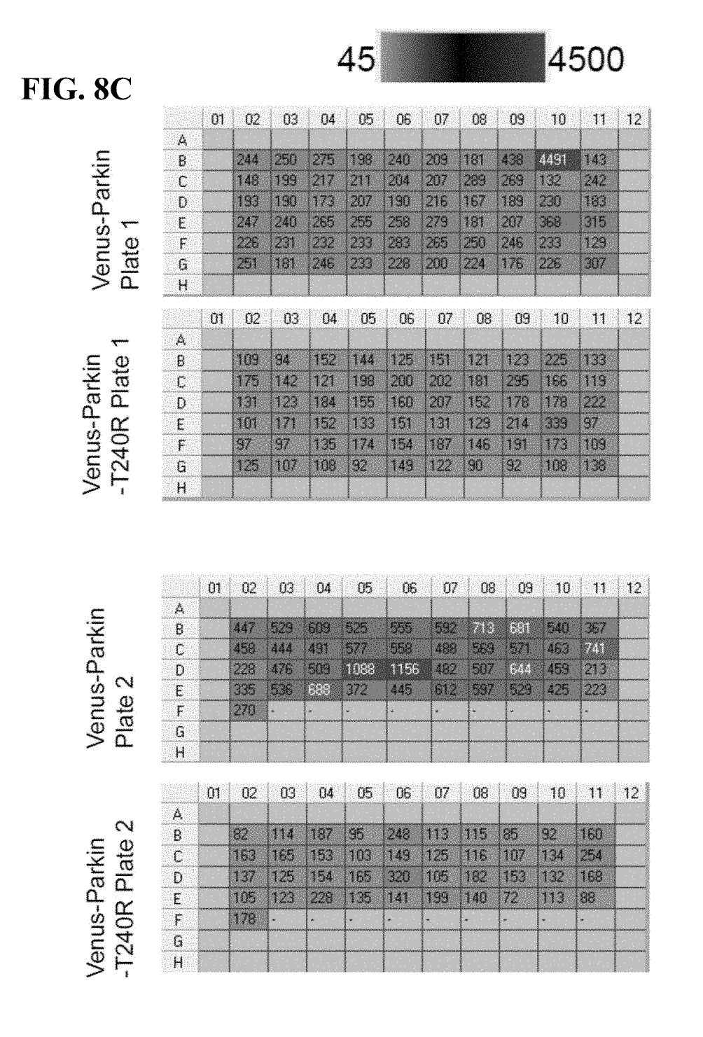

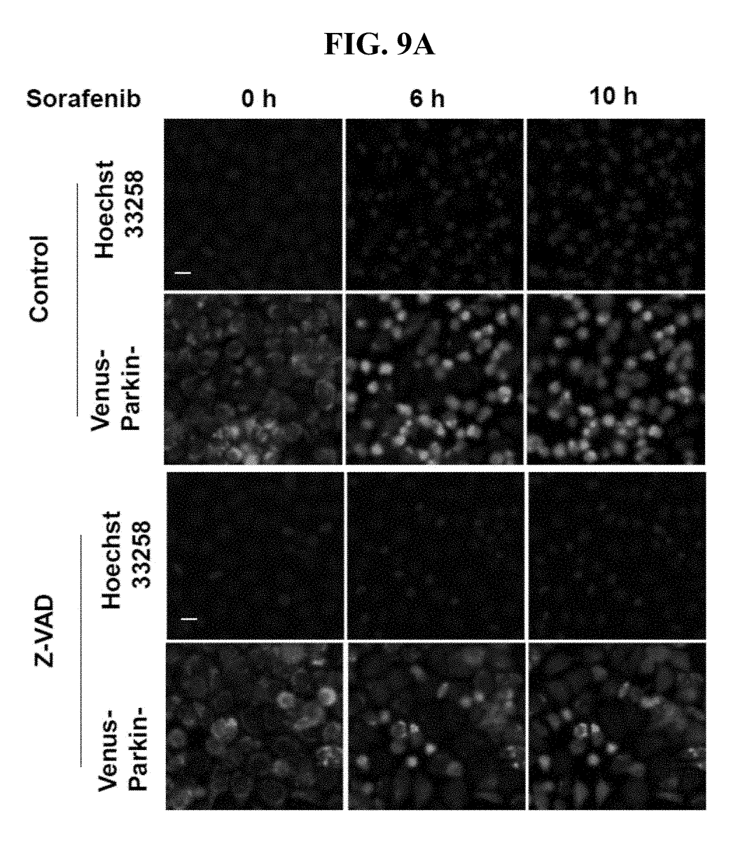

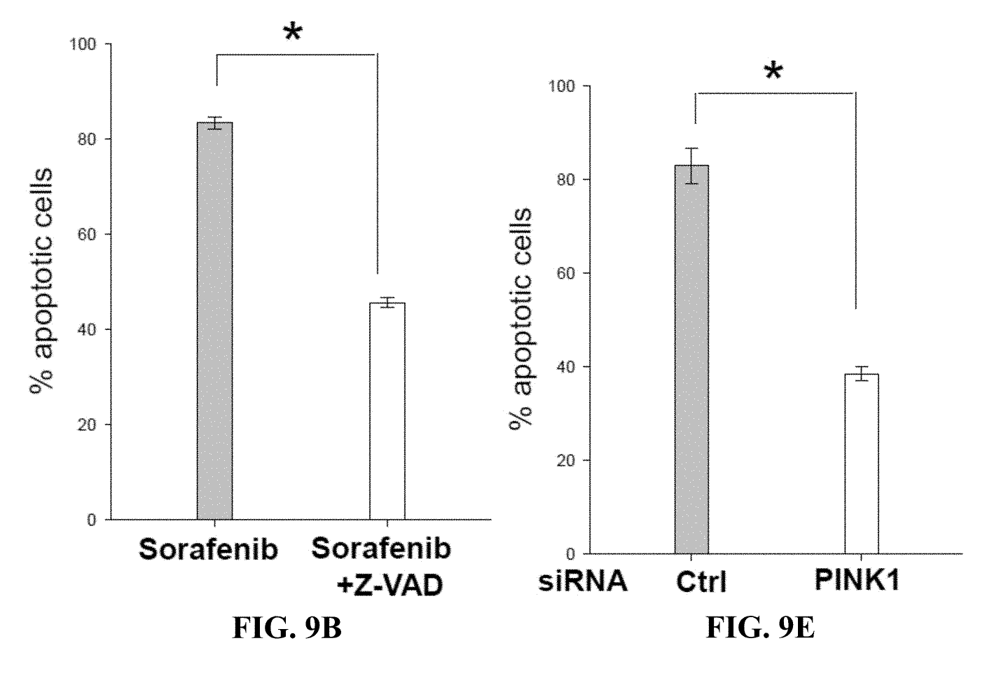

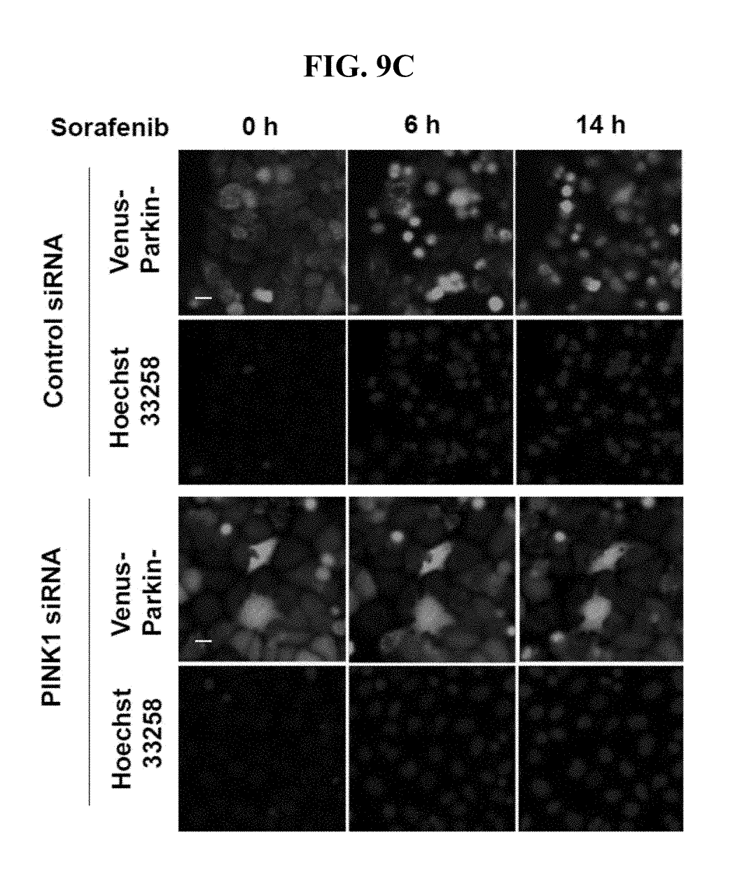

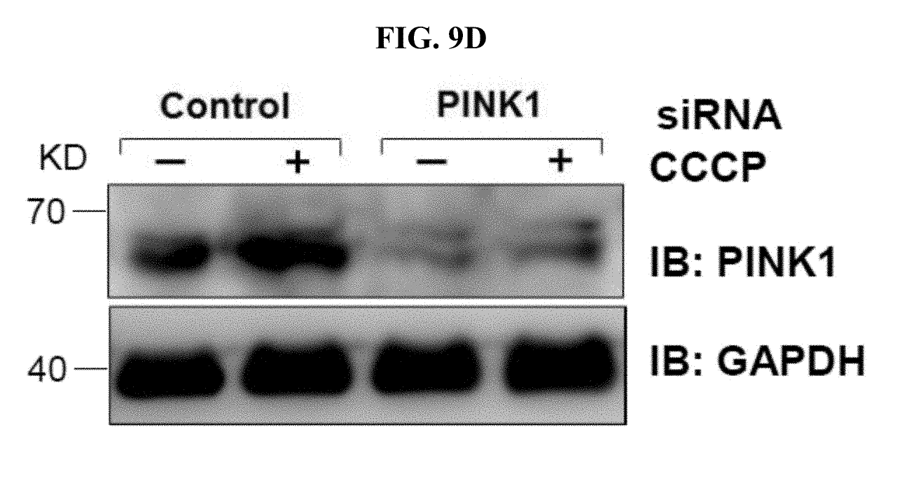

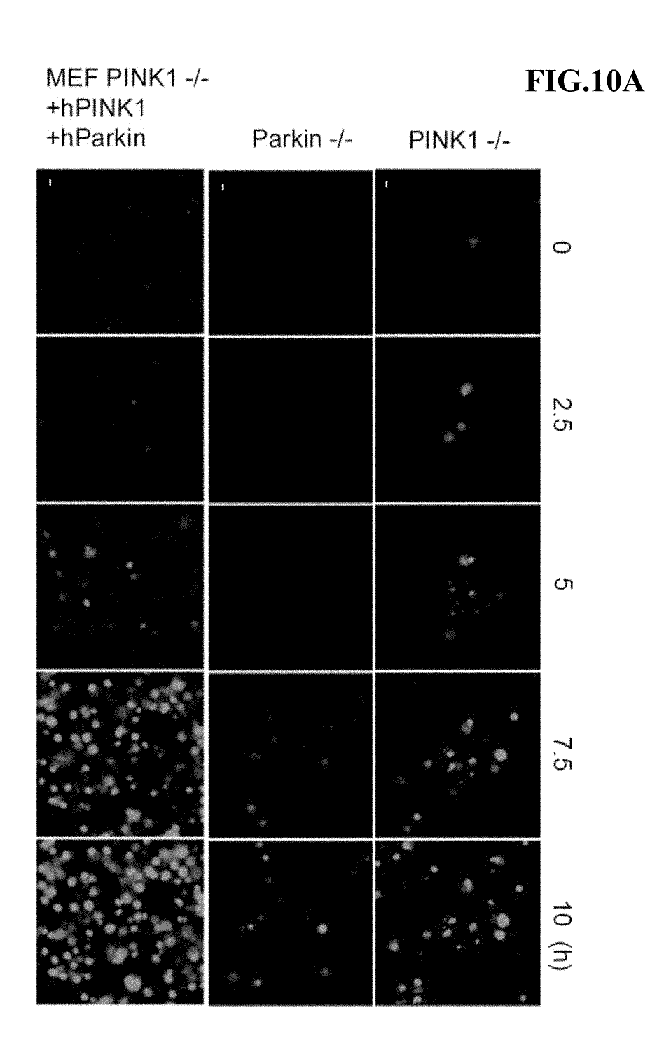

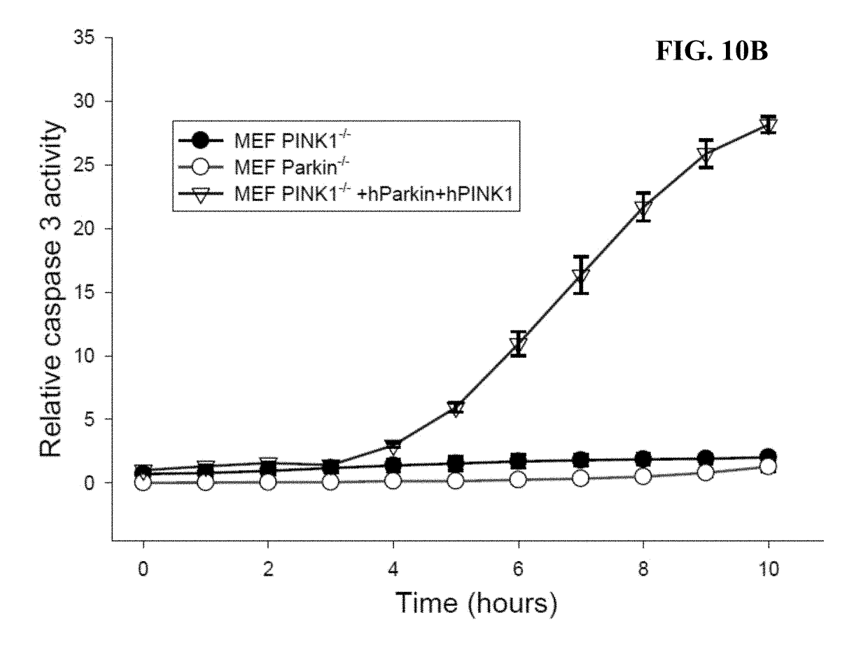

Sorafenib induces mitochondrial relocalization of Parkin: Previous studies have shown that Parkin relocates from cytosol to outer mitochondrial membrane in response to treatment with protonophore CCCP or potassium ionophore valinomycin but not rotenone or paraquat (Kondapalli, C., et al., (2012) PINK1 is activated by mitochondrial membrane potential depolarization and stimulates Parkin E3 ligase activity by phosphorylating Serine 65. Open Biol. 2, 120080). Our previous studies with HeLa cells stably expressing Venus-Parkin and RFP-Smac-MTS (mitochondrial targeting signal) demonstrated that CCCP and valinomycin trigger different cellular responses (Zhang, C., et al., Curr. Biol. 2014, supra). To further investigate differential cellular responses mediated by different chemicals, we performed a high content screening for Parkin mitochondrial recruitment response with the FDA approved oncology drug set (dtp.nci.nih.gov/branches/dscb/oncology_dr ugset_explanation.html). HeLa cells expressing RFP-Smac and VenusParkin-WT were treated with 20 μM compounds, and images of RFP-Smac and Venus-Parkin were collected at 1.5 h, 3 h, and 8 h using an HCS microscope ImageXpress (Molecular Devices). Parkin mitochondrial recruitment was quantified by a Transfluor-Colocalization Application Module (FIGS. 8A, 8B, 8C) which scores the relative colocalization efficiency of Parkin with mitochondria. As a control, HeLa cells stably expressing RFP-Smac-MTS and Venus-ParkinT240R, a patient-derived Parkin mutation that is defective in mitochondrial recruitment were also screened at the same conditions. The only condition that was capable of activating wild type Parkin from the screen was treatment of sorafenib. The effect of sorafenib on Parkin recruitment is specific, as Parkin T240R does not show mitochondrial recruitment under the same treatment. To further characterize Parkin mitochondrial recruitment, we compared the sorafenib response to that of a well-established trigger chemical, CCCP. As shown in FIGS. 1A, 1B, both 10 μM CCCP and 20 μM sorafenib could trigger Parkin to mitochondria. Time course studies showed Parkin translocation to mitochondria triggered by Sorafenib is slower than that triggered by CCCP (70 min delay) but reach maximum levels at about 2 hr (FIG. 10). Different doses of sorafenib were also tested in a time course experiment, and the result showed that the Parkin mitochondrial translocation induced by 25 μM sorafenib is comparable to 10 μM CCCP. Sorafenib-induced Parkin mitochondrial relocalization is PINK1 dependent—To test whether the sorafenib-induced Parkin localization to mitochondria is PINK1-dependent, we expressed Venus-Parkin in PINK1 knockout (PINK1 −/−) MEF cells. No Parkin mitochondrial translocation was detectable up to 40 μM sorafenib treatment. However, when a human PINK1 was introduced into this cell line, Venus-Parkin was rapidly recruited to damaged mitochondria in the presence of sorafenib (FIGS. 2A, 2C). Immunoblotting verified that sorafenib treatment induced PINK1 accumulation and Parkin auto-ubiquitination in MEF cells (FIG. 2B), indicating that sorafenib activates the PINK1/Parkin pathway in MEF cells. We have previously shown that PINK1 triggers autocatalytic activation of Parkin through phosphorylation at Ser 65 of the Ubl domain upon treatment with CCCP (Zhang, C., et al., Curr. Biol. 2014, supra). To determine whether sorafenib has a similar effect on Parkin activation, we treated HeLa cells stably expressing RFP-SmacMTS with Venus-Parkin-WT, mTurqoiseParkinS65A or both with 20 μM sorafenib. Immunoblotting showed that sorafenib only induced significant ubiquitination bands with wild type Venus-Parkin but not with mTurqoise-ParkinS65A in 2 h (FIG. 2D). mTurqoise-ParkinS65A mitochondrial recruitment was ineffective as compared with the wild type Parkin (FIG. 2E), suggesting PINK1 phosphorylation is required for Parkin activation. However, when Venus-ParkinWT and mTurqoise-ParkinS65A were co-expressed together in HeLa cells, sorafenib could trigger both proteins to mitochondria (FIGS. 2F, 2G, 2I). This data suggests Parkin is activated by an autocatalytic mechanism by sorafenib in a similar manner to CCCP. Sorafenib stabilizes PINK1 on the outer mitochondrial membrane and elevates PINK1 expression—Since PINK1 is required for sorafenib to induce Parkin mitochondrial recruitment, we next determined the effects of sorafenib on PINK1 expression and mitochondrial localization. First, we investigated whether sorafenib affects PINK1 expression by immunoblot in HeLa cells. As shown in FIG. 3A, sorafenib treatment induced a timedependent increase in the endogenous PINK1 levels in HeLa cells. The induction occurred as early as 1 hour after sorafenib addition. PINK1-EGFP when stably expressed in HeLa cells also increased upon sorafenib exposure (FIG. 3B). This result suggests that elevation of PINK1 is most likely due to posttranscriptional mechanism. Human PINK1 that is ectopically expressed in PINK1 null MEF cells showed a similar increase with sorafenib treatment. Sorafenib-dependent PINK1 elevation was observed in several cell lines including 293 and DLD1, suggesting that sorafenib-induced PINK1 elevation is well conserved in multiple cell lines and likely to be a general phenomenon. To determine whether sorafenib promotes PINK1 mitochondrial accumulation, we performed time-lapse imaging analysis of HeLa cells expressing PINK1-EGFP and RFP-Smac-MTS in the presence of 25 μM sorafenib. As shown in FIG. 3C, the level of PINK1-EGFP underwent steady increase until it levels off at 2 hours. Taken together, these results indicate that sorafenib stabilizes PINK1 on the outer membrane of mitochondria, which is responsible for subsequent Parkin mitochondrial accumulation. Dual inhibition of complex II/III of ETC and complex V by sorafenib is sufficient to activate Parkin recruitment to mitochondria-Parkin mitochondrial recruitment induced by CCCP has been attributed to depolarization of the mitochondrial membrane. To test whether sorafenib also triggers mitochondrial depolarization, HeLa cells were first labelled by a mitochondrial membrane potential chemical probe, tetramethylrhodamine methyl ester (TMRE), and exposed to DMSO, 10 μM CCCP, or 20 μM sorafenib individually for 40 minutes. DMSO treated cells showed slightly decrease in TMRE fluorescence over the time of the treatment. As expected, CCCP treatment caused a rapid decrease in fluorescence signal. Sorafenib exposure has a similar effect to CCCP, suggesting that sorafenib causes a loss of mitochondrial potential (FIG. 4A). As a proton ionophore, CCCP dissipates the proton gradient across the inner membrane of mitochondria. Sorafenib is not known to be an ionophore but inhibition of electron transport chain (ETC) could cause loss of Δψm. To test whether sorafenib inhibits ETC, we measured flavin adenine dinucleotide (FAD) autofluoresence by live cell confocal microscopy in the presence or absence of sorafenib or antimycin A, a known inhibitor of complex III followed by addition of CCCP (FIG. 4B). CCCP induces a sharp rise in FAD autofluorescence signal at 535 nm due to maximized respiration and oxidization of FADH2 with the uncoupling of ETC. Inhibition of complex III by antimycin A blocks electron transport and FADH2 electron loss thereby suppressing the FAD autofluoresence signal. Short exposure to sorafenib resulted in a similar suppression of FAD production, suggesting sorafenib may be an inhibitor of electron transport chain. To identify the mitochondrial targets of sorafenib, we systematically measured the inhibitory activity of sorafenib toward all electron transport chain complexes and complex V using immuno-captured complexes on the microplate or isolated mitochondria (complex II/III). As shown in FIG. 4, sorafenib showed inhibition of complex II/III and complex V at 20 μM and little if any activity against complex I and complex IV (FIGS. 4C, 4D, 4E, 4F). The inhibitory activity of sorafenib towards complex II/III and complex V were further evaluated through dose response analysis. The IC50 values are ˜10 μM and ˜2.5 μM for complex II/III and complex V, respectively (FIGS. 4G, 4H). To elucidate the significance of dual inhibition of ETC by sorafenib in activating PINK1-Parkin, we tested well established selective inhibitors of ETC individually on Parkin recruitment and found that none of them can activate Parkin at 10 μM, which is in agreement with previous results (Kondapalli, C., et al., (2012, supra). For example, neither antimycin A, a complex II/III inhibitor, nor oligomycin, a complex V inhibitor alone triggers Parkin mitochondrial recruitment (FIGS. 4I, 4J). However, when antimcyin A was added together with oligomycin, a robust Parkin localization was observed. This result suggests that sorafenib is essentially the sum of antimycin plus oligomycin and activation of PINK1-Parkin is most likely due to dual inhibition of complex II/III and complex V. Next, we determined the effects of sorafenib or combination of ETC complexes inhibitor on the mitochondrial membrane potentials. As shown in FIG. 4J, CCCP or sorafenib alone caused a rapid reduction in TMRE fluorescence, indicative of Δψm loss. As expected, oligomycin alone resulted in hyperpolarization of the mitochondrial membrane potential due to the reduction of proton flow through complex V. KCN alone caused a minor reduction in TMRE signal and antimycin at 10 μM caused a moderate reduction in TMRE (FIG. 4K). However, the combination of antimycin and oligomycin resulted in a signal loss similar to CCCP or sorafenib. Combination of KCN with oligomycin also resulted in significant TMRE signal loss. However, this combination does not activate Parkin mitochondria recruitment (FIG. 4I). Collectively, our results suggest that sorafenib or antimycin plus oligomycin most likely causes depolarization of mitochondrial membrane potential via dual inhibition of complex II/III and V which subsequently engenders PINK1 stabilization at mitochondrial outer membrane and Parkin recruitment. While depolarization of mitochondrial membrane potential is necessary for activation of this pathway, loss of Δψm is not sufficient to cause robust pathway activation. Sorafenib induces PINK1/Parkin-dependent apoptosis-Since the PINK1/Parkin pathway has been shown to mediate distinct cellular outcomes in response to different stress stimuli (Zhang, C., et al., Curr. Biol. 2014, supra), we tested the effect of sorafenib exposure on cell fate determination in HeLa cells stably expressing Venus-Parkin-WT, RFP-Smac-MTS and CFP-LC3 (Zhang, C., et al., Curr. Biol. 2014, supra). In agreement with previous results, CCCP treatment induced Parkin mitochondrial accumulation followed by mitophagy as determined by mitochondrial clustering and CFPLC3 mitochondrial co-localization (FIGS. 5A, 5C, 5D). In contrast, sorafenib treatment elicited a strong apoptotic response, quantified by RFP-Smac-MTS release from mitochondria i.e. mitochondrial outer membrane permeability (MOMP) (FIGS. 5B, 5C, 5D). Independently we also measured sorafenib-induced apoptosis response by Hoechst 33258 fluorescence using an automated MetaXpress application module (FIGS. 9A 9B). Increased nuclei condensation state as seen by Hoechst staining intensity in the presence of sorafenib further confirmed the pro-apoptotic effects. Additionally, increased Hoechst staining by sorafenib treatment was abrogated by the presence of Z-VAD-FMK, a caspase inhibitor. This result further supports that sorafenib induces caspase-dependent apoptotic Next, we sought to determine whether the PINK1/Parkin pathway plays a role in sorafenibinduced apoptosis. The fact that the parental HeLa cells do not express Parkin allowed us to compare the sorafenib response with or without functional Parkin expression. HeLa cells expressing RFPSmac-MTS with Venus-Parkin-WT or WenusParkin-T240R were incubated with 30 μM sorafenib for 7.5 hours (FIGS. 5E, 5F). The apoptosis response was quantified by counting RFP-Smac release from mitochondria, a reporter of MOMP. Venus-Parkin-WT cells exposed to sorafenib clearly display enriched mitochondria parkin localization and apoptosis, whereas cells expressing Venus-Parkin-T240R under the same conditions have almost no Parkin translocation to mitochondria and cell death (FIGS. 5E, 5F). Time-dependent apoptotic response was measured and quantified for the parental HeLa cells, Venus-Parkin and Venus Parkin T240R HeLa cells (FIGS. 5G, 4H). HeLa cells with wild type Parkin showed higher rates of apoptosis compared to the parental or Parkin T240R mutant, suggesting that sorafenib-induced apoptosis is Parkin dependent. To test if the sorafenib-induced apoptosis is also PINK1dependent, PINK1 was depleted using the PINK1 siRNA. As shown in FIGS. 9C, 9D, 9E, PINK1 knockdown blocked sorafenib-induced translocation of Parkin to mitochondria and significantly decreased rate of apoptosis (FIGS. 9C, 9E). We also investigated that the endogenous PINK1/Parkin pathway also regulates sorafenib apoptotic response in other cell lines. Mouse embryonic fibroblast cell lines (MEFs) that were derived from PINK1 or Parkin knockout or with wild type mice were treated with 30 μM sorafenib and apoptosis was quantified by NucView caspase-3 sensor and Hoechst 33258 staining. There was less apoptosis in PINK1 and Parkin null MEF cells than in their wild type counterparts (FIGS. 10A, 10B), suggesting that loss of either PINK1 or Parkin makes cells less sensitive to induction of apoptosis by sorafenib treatment. The defects in sorafenib response could be fully restored in these knockout cell lines upon expression of human PINK1 or Parkin. Collectively, these results indicate that PINK1 and Parkin is involved in regulating sorafenib-induced cellular apoptosis. Sorafenib induces PINK1/Parkin-dependent apoptosis by suppressing Bcl-2 family of proteins. Both CCCP and sorafenib cause PINK1 induction and mitochondrial localization of Parkin, yet the biological outcomes are distinct (mitophagy vs. apoptosis). We sought to determine the molecular basis for the divergent responses for these two compounds. First, we compared caspase-3 and PARP activation in wild type and Parkin mutant cell lines in response to sorafenib and CCCP treatments by immunoblotting. Consistent with apoptotic response induced by sorafenib, procaspase-3 was reduced and PARP cleavage was strongly induced by sorafenib but not CCCP in a Parkin-dependent manner (FIGS. 6A, 6B). We and others have previously shown that Parkin can target Bcl-2 family of proteins to regulate apoptosis (Zhang, C., et al., Curr. Biol. 2014, supra; Carroll, R. G., et al., (2014) Parkin Sensitizes toward Apoptosis Induced by Mitochondrial Depolarization through Promoting Degradation of Mcl-1. Cell Rep. 9:1538-53). To determine whether sorafenib or CCCP regulate Bcl-2 family protein, we blotted Bcl-2, Mcl-1, and Bcl-xL in HeLa with wild type or mutant Parkin. Sorafenib strongly suppressed expression of Mcl-1 in a Parkin-dependent manner but induced no significant changes in expression levels for Bcl-2 and Bcl-xL (FIGS. 6C, 6D). In contrast, no significant changes were seen in the three Bcl-2 family members with CCCP treatment in either cell lines (FIGS. 6C, 6D). To determine whether CCCP and sorafenib may activate PINK1 and Parkin differently in HeLa cells, we also blotted PINK1 and Parkin upon treatment with sorafenib and CCCP in parallel in HeLa cells expressing Venus-Parkin. PINK1 elevation occurred with both treatments, while drastic Mcl-1 down-regulation was only seen in sorafenib-treated cells (FIG. 6E). Suppression of Mcl-1 can occur through a transcriptional mechanism or posttranscriptional mechanism. To determine if proteasomal is involved in Mcl-1 suppression by sorafenib, MG132 was added along with sorafenib and compared to treatment with sorafenib alone. Inhibition of the proteasome completely abrogated suppression of Mcl-1 by sorafenib (FIG. 6F), and higher molecular weight species appeared with MG-132 treatment, which may correspond to ubiquitylated but not degraded Mcl-1. This result suggests that down-regulation of Mcl-1 by sorafenib largely occurs through degradation by the ubiquitin/proteasome pathway. Since Mcl-1 suppression by sorafenib also required functional Parkin (FIG. 6C), we conclude that sorafenib activates the PINK1-Parkin pathway to degrade Mcl-1. To test the role of Mcl-1 degradation in the apoptotic response of cells to sorafenib, we knocked down the expression of Mcl-1 with a shRNA expression vector in HeLa cells with reconstituted Parkin. Immunoblotting experiments confirmed reduction of Mcl-1 (FIG. 6G). Timelapse imaging of the control or Mcl-1 knockdown HeLa cells treated with CCCP or sorafenib revealed that Mcl-1 knockdown accelerated the sorafenib induced apoptotic response, and, more strikingly, the CCCP-induced mitophagy response was switched to an apoptotic response (FIG. 6H). Therefore, the cellular decision to undergo apoptosis or mitophagy depends not only on activation of Parkin on mitochondria, but also on the Mcl-1 levels within the cell. If Parkin functions upstream of the Bcl2 family proteins to modulate responses to sorafenib, we would expect that high levels of Bcl-2 proteins may antagonize sorafenib induced Parkin dependent apoptotic response. HeLa cells have significant Mcl-1 levels, and introduction of Mcl-1 did not result in significant overexpression of Mcl1 (data not shown). However, we were able to overexpress CFP-tagged mouse Bcl-2 in HeLa cells (˜50 kD) (FIG. 6I). In HeLa cells stably expressing mBcl-2, there was significant reduction in both the percentage of cells (˜50%) that underwent apoptosis and delayed triggering apoptosis upon treatment with sorafenib (FIG. 6J). Overexpression of Bcl2 effectively suppresses the activity of Parkin in HeLa cells and this result supports our model that Parkin acts upstream of Bcl2 family proteins to regulate drug sensitivity to sorafenib. Rationalized drug combinations to shift autophagy response to apoptotic response-Results shown in FIGS. 6I, 6J suggest that overexpression of Bcl-2 makes cells more resistant to sorafenibinduced cellular apoptosis. The Bcl-2 family of proteins act downstream of PINK1 and Parkin to dictate the Parkin-dependent apoptotic responses to sorafenib. Based on this result, we hypothesized that lower anti-apoptotic activity of Bcl-2 should make cells more sensitive to sorafenib. ABT-737 is a BH3 mimetic inhibitor of Bcl-xL, Bcl-2, and Bclw with EC50 of 78.7 nM, 30.3 nM, and 197.8 nM, respectively (Selleckchem). No inhibition has been observed against Mcl-1, Bcl-B, or Bfl-1. To test our hypothesis, HeLa cells with or without Bcl-2 overexpression were treated with sorafenib, ABT737, or both in combination. HeLa mBcl-2 cell lines were more resistant to sorafenib, as expected. ABT737 had no single-agent apoptotic activity at 10 μM for either cell line. However, when sorafenib and ABT-737 were added together, both the wild type and the Bcl2 overexpressing cells were completely eliminated by apoptosis (FIG. 7A). This result suggests that the combination of the bcl-2 inhibitor ABT-737 with sorafenib synergistically triggers robust apoptosis of cancer cells. We also tested the combination effects of CCCP and ABT-737, since neither of them alone had significant cell killing activity. When CCCP and ABT-737 were added together, robust apoptotic activity was observed (FIG. 7B). This result suggests that cancer cells with a robust PINK1-Parkin pathway are more susceptible to combined treatment with sorafenib and Bcl-2 inhibitors.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiments described hereinabove are further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

1-4. (canceled)

5. A method of selectively treating cancer comprising:
(a) selecting a patient for treatment with an anti-cancer agent on the basis of the patient having, in a cancer cell, the level of a biomarker selected from:
a) PINK1;
b) Parkin;
c) Mcl-1;
d) Bcl-2;
and,
(b) selectively administering the anti-cancer agent to the patient.

6. (canceled)

7. A method for predicting the clinical response of a human cancer patient to an anti-cancer medication comprising:
obtaining a cancer cell from a patient diagnosed with cancer, the cancer cell comprising nucleic acids from the patient;
detecting in the nucleic acids the level of a biomarker selected from:
a) PINK1;
b) Parkin;
c) Mcl-1;
d) Bcl-2;
and,
correlating the level of the biomarker with an increased likelihood for the patient to have a beneficial clinical response to an anti-cancer medication.

8. The method of claim 5, wherein the anti-cancer medication inhibits the activity of both complex II/III and complex V of electron transport chain.

9. The method of claim 5, wherein the anti-cancer medication is a tyrosine kinase inhibitor, and an inhibitor of the activity of both complex II/III and complex V of electron transport chain.

10. The method of claim 5, wherein the anti-cancer medication is sorafenib.

11. The method of claim 5, wherein the anti-cancer medication is selected from the group consisting of sorafenib, regorafenib, ABT-737, doxorubicin, cetuximab, valinomycin, and CCCP.

12. The method of claim 5, wherein the anti-cancer medication is at least one of sorafenib and regorafenib.

13. The method of claim 5, wherein the anti-cancer medication is at least one of sorafenib and regorafenib combined with at least one of ABT-737, doxorubicin, and cetuximab.

14. The method of claim 5, wherein the anti-cancer medication is at least one of valinomycin and CCCP.

15. The method of claim 5, wherein the anti-cancer medication is valinomycin.

16. The method of claim 5, wherein the anti-cancer medication is CCCP.

17. The method of claim 5, wherein the benefit is selected from anticancer response to the compound, better disease control rate, longer time to progression and increased survival following treatment with the anticancer compound.

18. The method of claim 5, wherein the cancer cell is one of a hepatocellular carcinoma cell, a renal cell carcinoma cell, a pancreatic carcinoma cell, a glioblastoma cell, a colorectal cancer (CRC) cell, a chemotherapy-refractory metastatic CRC cell, and a late-stage metastatic CRC cell.

19. The method of claim 5, wherein the biomarker is detected by a method selected from Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry.

20. The method of claim 5, wherein the biomarker is detected by immunohistochemical (IHC) analysis.

21. An assay kit for selecting a cancer patient who is predicted to benefit or not to benefit from therapeutic administration of an anticancer compound, the assay kit comprising:
i) a means for detecting in a sample of cancer cells a level of a biomarker or a combination of biomarkers selected from the group consisting of:
a) high expression levels of PINK1;
b) high expression levels of Parkin;
c) low expression levels of Mcl-1;
d) low expression levels of Bcl-2;
e) observed mitochondrial localization of wild type Parkin following treatment with sorafenib or regorafenib, in combination with low Mcl-1 or Bcl-2 expression;
and,
ii) a control selected from the group consisting of:
a) a control sample for detecting sensitivity to the anticancer compound;
b) a control sample for detecting resistance to the anticancer compound;
c) information containing a predetermined control level of the biomarker that has been correlated with sensitivity to the anticancer compound; and
d) information containing a predetermined control level of the biomarker that has been correlated with resistance to the anticancer compound.

22-25. (canceled)
